(19) United States
(12) Patent Application Publication
(10) Pub. No.: US 2010/0324123 A1
(43) Pub. Date: Dec. 23, 2010

(54) GLMS RIBOSWITCHES, STRUCTURE-BASED COMPOUND DESIGN WITH GLMS RIBOSWITCHES, AND METHODS AND COMPOSITIONS FOR USE OF AND WITH GLMS RIBOSWITCHES

(75) Inventors: Ronald R. Breaker, Guilford, CT (US); Jinsoo Lim, Lexington, MA (US); Scott A. Strobel, Hamden, CT (US); Jesse C. Cochrane, New Haven, CT (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Yale University, New Haven, CT (US)

(21) Appl. No.: 12/440,172

(22) PCT Filed: Sep. 6, 2007
(86) PCT No.: PCT/US07/19456

§ 371 Date: Aug. 25, 2010

Related U.S. Application Data
(60) Provisional application No. 60/842,870, filed on Sep. 6, 2006.
(60) Provisional application No. 60/844,844, filed on Sep. 16, 2006.

(51) Int. Cl. A61K 31/7105; C12N 1/20; A61K 31/70; A61K 31/7008; C07H 5/04; C07H 1/00; C12Q 1/68; C07H 21/02; A61P 31/04; A01P 1/00; A01N 57/10

(52) U.S. Cl. 514/44 R; 435/252.1; 514/23; 536/18.7; 536/1.11; 435/6; 536/24.1

(57) ABSTRACT

The glmS riboswitch is a target for antibiotics and other small molecule therapies. Compounds can be used to stimulate, active, inhibit and/or inactivate the glmS riboswitch. The atomic structures of the glmS riboswitch can be used to design new compounds to stimulate, active, inhibit and/or inactivate riboswitches.

### CROSS-REFERENCE TO RELATED APPLICATIONS

This application claims benefit of U.S. Provisional Application No. 60/842,870, filed Sep. 6, 2006 and U.S. Provisional Application No. 60/844,844, filed Sep. 16, 2006. U.S. Provisional Application No. 60/842,870, filed Sep. 6, 2006 and U.S. Provisional Application No. 60/844,844,. filed Sep. 16, 2006, are hereby incorporated herein by reference in their entirety.

### STATEMENT REGARDING FEDERALLY SPONSORED RESEARCH

This invention was made with government support under Grant Nos. MCB 0544255 and EIA-032351 awarded by the National Science Foundation; Grant No. W911NF-04-1-0416 awarded by DARPA; and Grant NIH R33-DK070270 awarded by the NIH. The government has certain rights in the invention.

### FIELD OF THE INVENTION

The disclosed invention is generally in the field of gene expression and specifically in the area of regulation of gene expression.

### BACKGROUND OF THE INVENTION

Precision genetic control is an essential feature of living systems, as cells must respond to a multitude of biochemical signals and environmental cues by varying genetic expression patterns. Most known mechanisms of genetic control involve the use of protein factors that sense chemical or physical stimuli and then modulate gene expression by selectively interacting with the relevant DNA or messenger RNA sequence. Proteins can adopt complex shapes and carry out a variety of functions that permit living systems to sense accurately their chemical and physical environments. Protein factors that respond to metabolites typically act by binding DNA to modulate transcription initiation (e.g. the lac repressor protein; Matthews, K. S., and Nichols, J. C., 1998, Prog. Nucleic Acids Res. Mol. Biol. 58, 127-164) or by binding RNA to control either transcription termination (e.g. the PyrR protein; Switzer, R. L., et al., 1999, Prog. Nucleic Acids Res. Mol. Biol. 62, 329-367) or translation (e.g. the TRAP protein; Babitzke, P., and Gollnick, P., 2001, J. Bacteriol. 183, 5795-5802). Protein factors respond to environmental stimuli by various mechanisms such as allosteric modulation or post-translational modification, and are adept at exploiting these mechanisms to serve as highly responsive genetic switches (e.g. see Ptashne, M., and Gann, A. (2002). Genes and Signals. Cold Spring Harbor Laboratory Press, Cold Spring Harbor, N.Y.).

In addition to the widespread participation of protein factors in genetic control, it is also known that RNA can take an active role in genetic regulation. Recent studies have begun to reveal the substantial role that small non-coding RNAs play in selectively targeting mRNAs for destruction, which results in down-regulation of gene expression (e.g. see Hannon, G. J. 2002, Nature 418, 244-251 and references therein). This process of RNA interference takes advantage of the ability of short RNAs to recognize the intended mRNA target selectively via Watson-Crick base complementation, after which the bound mRNAs are destroyed by the action of proteins. RNAs are ideal agents for molecular recognition in this system because it is far easier to generate new target-specific RNA factors through evolutionary processes than it would be to generate protein factors with novel but highly specific RNA binding sites.

Although proteins fulfill most requirements that biology has for enzyme, receptor and structural functions, RNA also can serve in these capacities. For example, RNA has sufficient structural plasticity to form numerous ribozyme domains (Cech & Golden, Building a catalytic active site using only RNA. In: The RNA World R. F. Gesteland, T. R. Cech, J. F. Atkins, eds., pp. 321-350 (1998); Breaker, In vitro selection of catalytic polynucleotides. Chem. Rev. 97, 371-390 (1997)) and receptor domains (Osborne & Ellington, Nucleic acid selection and the challenge of combinatorial chemistry. Chem. Rev. 97, 349-370 (1997); Hermann & Patel, Adaptive recognition by nucleic acid aptamers. Science 287, 820-825 (2000)) that exhibit considerable enzymatic power and precise molecular recognition. Furthermore, these activities can be combined to create allosteric ribozymes (Soukup & Breaker, Engineering precision RNA Molecular switches. Proc. Natl. Acad. Sci. USA 96, 3584-3589 (1999); Seetharaman et al., Immobilized riboswitches for the analysis of complex chemical and biological mixtures. Nature Biotechnol. 19, 336-341 (2001)) that are selectively modulated by effector molecules.

Bacterial riboswitch RNAs are genetic control elements that are located primarily within the 5′-untranslated region (5′-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). What is needed in the art are methods and compositions that can be used to regulate glmS riboswitches.

### BRIEF SUMMARY OF THE INVENTION

It has been discovered that certain natural mRNAs serve as metabolite-sensitive genetic switches wherein the RNA directly binds a small organic molecule. This binding process changes the conformation of the mRNA, which causes a change in gene expression by a variety of different mechanisms. The natural switches are targets for antibiotics and other small molecule therapies.

Disclosed are compounds, and compositions containing such compounds, that can activate, deactivate or block the glmS riboswitch. Also disclosed are compositions and methods for activating, deactivating or blocking the glmS riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. A riboswitch can be blocked by, for example, binding of an analog of the trigger molecule that does not activate the riboswitch.

Also disclosed are compositions and methods for altering expression of an RNA molecule, or of a gene encoding an RNA molecule, where the RNA molecule includes a glmS riboswitch, by bringing a compound into contact with the RNA molecule. Riboswitches function to control gene expression through the binding or removal of a trigger molecule. Thus, subjecting an RNA molecule of interest that includes a glmS riboswitch to conditions that activate, deactivate or block the riboswitch can be used to alter expression of the RNA. Expression can be altered as a result of, for example, termination of transcription or blocking of ribosome binding to the RNA. Binding of a trigger molecule or an analog thereof can, depending on the nature of the riboswitch, reduce or prevent expression of the RNA molecule or promote or increase expression of the RNA molecule.

Also disclosed are compositions and methods for regulating expression of a naturally occurring gene or RNA that contains a glmS riboswitch by activating, deactivating or blocking the riboswitch. If the gene is essential for survival of a cell or organism that harbors it, activating, deactivating or blocking the glmS riboswitch can result in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects.

Disclosed herein is a compound having the structure of Formula I:

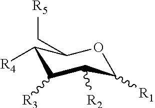

or pharmaceutically acceptable salts thereof, physiologically hydrolyzable and acceptable esters thereof, or both, wherein R₁ is H, OH, SH, NH₂, or CH₃, wherein R₂ is NH—R₆, wherein R₆ is H, CH₃, C₂H₅, n-propyl, C(O)CH₃, C(O)C₂H₅, C(O)n-propyl, C(O)iso-propyl, C(O)OCH₃, C(O)OC₂H₅, C(O)NH₂, or NH₂, wherein R₃ is H, OH, SH, NH₂, or CH₃, wherein R₄ is a hydrogen bond donor, wherein R₅ is a hydrogen bond acceptor, and wherein the compound is not glucosamine-6-phosphate. Also disclosed are compounds in which R₄ is OH, SH, NH₂, NH₃+, CH₂OH, CH(OH)CH₃, CH₂CH₂OH, CH₂SH, CH(SH)CH₃, CH₂CH₂SH, CH₂NH₂, CH(NH₂)CH₃, CH₂CH₂NH₃, CO₂H, CONH₂, CONHalkyl, ═NH, ═NOH, ═NSH, ═NCO₂H, ═CH₂, CH═NH, CH═NOH, CH═NSH, CH═NCO₂H, OCH₂OH, OCH₂CH₂OH, PhOH, NHalkyl, NHNH₂, NHNHalkyl, NHCOalkyl, NHCO₂alkyl, NHCONH₂, NHSO₂alkyl, or NHOalkyl. Also disclosed are compounds in which R₄ is not OH when R₁ is H or OH and R₂ is NH₂ or NHCH₃. Further disclosed are compounds in which R₅ is OP(O)(OH)₂, OP(S)(OH)₂, OP(O)OHSH, OS(O)₂OH, or OS(O)₂SH. Also disclosed are compounds in which R₅ is OS(O)₂OH or OS(O)₂SH. Furthermore, R₅ can be negatively charged. Further disclosed are compounds in which R₅ is ═O, CO₂R₉, OCO₂R₉, OCH₂OR₉, OC₂H₅OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, PO₂(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, and wherein R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), —C(CH₃)₃, or —CF₃. Also disclosed are compounds in which R₅ is ═O, OH, OR₉, COR₉, CN, NO₂, tetrazole, SOR₉, N(R₉)₂, CO₂R₉, OCO₂R₉, OCH₂OR₉, OC₂H₅OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, PO₂(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, and R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), —C(CH₃)₃, or —CF₃. Also disclosed are compounds in which R₄ is NH₂, NH₃⁺, OH, SH, NOH, NHNH₂, NHNH₃⁺, CO₂H, SO₂OH, B(OH)₂, or imidazolium. Also disclosed are compounds in which R₄ is NH₂, NH₃⁺, SH, NOH, NHNH₂, NHNH₃⁺, CO₂H, SO₂OH, B(OH)₂, or imidazolium. The compounds disclosed above can activate a glmS-responsive riboswitch.

Also disclosed herein is a method of inhibiting gene expression, the method comprising bringing into contact a compound as disclosed above and a cell, wherein the cell comprises a gene encoding an RNA comprising a glmS-responsive riboswitch, wherein the compound inhibits expression of the gene by binding to the glmS-responsive riboswitch. The cell can be a bacterial cell, for example, and the compound can kill or inhibit the bacterial cell. The cell can contain a glmS riboswitch. The cell can be Bacillus or Staphylococcus.

Also disclosed is a method of inhibiting gene expression in a cell and/or inhibiting cell growth in a subject containing the cell by bringing into contact a compound and the cell by administering the compound to a subject. In some forms of the method, the compound is not a substrate for enzymes of the subject that have glucosamine-6-phosphate as a substrate. In some forms of the method, the compound is not a substrate for enzymes of the subject that alter glucosamine-6-phosphate. In some forms of the method, the compound is not a substrate for enzymes of the subject that metabolize glucosamine-6-phosphate. In some forms of the method, the compound is not a substrate for enzymes of the subject that catabolize glucosamine-6-phosphate. In some forms of the method, the cell is a bacterial cell in the subject, wherein the compound kills or inhibits the growth of the bacterial cell. In some forms of the method, the subject has a bacterial infection. In some forms of the method, the compound is administered in combination with another antimicrobial compound. In some forms of the method, the compound inhibits bacterial growth in a biofilm.

Further disclosed is a composition comprising the compound described above and a regulatable gene expression construct comprising a nucleic acid molecule encoding an RNA comprising a glmS riboswitch operably linked to a coding region, wherein the glmS riboswitch regulates expression of the RNA, wherein the glmS riboswitch and coding region are heterologous. The glmS riboswitch can produce a signal when activated by the compound. For example, the riboswitch can change conformation when activated by the compound, and the change in conformation can produce a signal via a conformation dependent label. Furthermore, the riboswitch can change conformation when activated by the compound, wherein the change in conformation causes a change in expression of the coding region linked to the riboswitch, wherein the change in expression produces a signal. The signal can be produced by a reporter protein expressed from the coding region linked to the riboswitch.

Also disclosed is a method comprising: (a) testing the compound as described above for inhibition of gene expression of a gene encoding an RNA comprising a glmS riboswitch, wherein the inhibition is via the glmS riboswitch, and (b) inhibiting gene expression by bringing into contact a cell and a compound that inhibited gene expression in step (a), wherein the cell comprises a gene encoding an RNA comprising the glmS riboswitch, wherein the compound inhibits expression of the gene by binding to the glmS riboswitch.

Also disclosed are the crystalline atomic structures of riboswitches. For example, disclosed is the atomic structure of a natural glmS-responsive riboswitch comprising an atomic structure comprising the atomic coordinates listed in Table 2, the atomic structure of the active site and binding pocket as depicted in FIG. 9, and the atomic coordinates of the active site and binding pocket depicted in FIG. 9 contained within Table 2. The atomic coordinates of the binding pocket depicted in FIG. 9 contained within Table 2 is referred to herein as the binding pocket atomic structure. The atomic coordinates of the active site depicted in FIG. 9 contained within Table 2 is referred to herein as the active site atomic structure. These structures are useful in modeling and assessing the interaction of a riboswitch with a binding ligand. They are also useful in methods of identifying compounds that interact with the riboswitch. Any useful portion of the structure can be used for purposed and modeling as described herein. In particular, the active site or binding pocket atomic structure, with or without additional surrounding structure, cona be modeled and used in the disclosed methods.

Also disclosed are methods of identifying a compound that interacts with a riboswitch comprising modeling the atomic structure of the riboswitch with a test compound and determining if the test compound interacts with the riboswitch. This can be done by determining the atomic contacts of the riboswitch and test compound. Furthermore, analogs of a compound known to interact with a riboswitch can be generated by analyzing the atomic contacts, then optimizing the atomic structure of the analog to maximize interaction. These methods can be used with a high throughput screen.

Further disclosed is a method of identifying a compound that interacts with a riboswitch comprising: modeling the atomic structure of a glmS riboswitch with a test compound; and determining if the test compound interacts with the riboswitch. Furthermore, determining if the test compound interacts with the riboswitch can comprise determining a predicted minimum interaction energy, a predicted binding constant, a predicted dissociation constant, or a combination, for the test compound in the model of the riboswitch. Determining if the test compound interacts with the riboswitch can comprise determining one or more predicted bonds, one or more predicted interactions, or a combination, of the test compound with the model of the riboswitch. Atomic contacts can be determined, thereby determining the interaction of the test compound with the riboswitch. The method of identifying a compound that interacts with a riboswitch can further comprise the steps of identifying analogs of the test compound; and determining if the analogs of the test compound interact with the riboswitch.

Further disclosed is a method of killing or inhibiting the growth of bacteria, comprising contacting the bacteria with a compound identified by the method disclosed above. Further disclosed is a method of killing bacteria, comprising contacting the bacteria with a compound identified by the method disclosed above. A gel-based assay or a chip-based assay can be used to determine if the test compound interacts with the riboswitch. The test compound can interact via van der Waals interactions, hydrogen bonds, electrostatic interactions, hydrophobic interactions, or a combination. The riboswitch can comprise an RNA cleaving ribozyme, for example. A fluorescent signal can be generated when a nucleic acid comprising a quenching moiety is cleaved. Molecular beacon technology can be employed to generate the fluorescent signal. The methods disclosed herein can be carried out using a high throughput screen.

Also disclosed are compositions and methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Deactivation of a riboswitch refers to the change in state of the riboswitch when the trigger molecule is not bound. A riboswitch can be deactivated by binding of compounds other than the trigger molecule and in ways other than removal of the trigger molecule. Blocking of a riboswitch refers to a condition or state of the riboswitch where the presence of the trigger molecule does not activate the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Disclosed herein is also a method of inhibiting growth of a cell, such as a bacterial cell, that is in a subject, the method comprising administering an effective amount of a compound as disclosed herein to the subject. This can result in the compound being brought into contact with the cell. The subject can have, for example, a bacterial infection, and the bacterial cells can be the cells to be inhibited by the compound. The bacteria can be any bacteria, such as bacteria from the genus Bacillus or Staphylococcus, for example. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows structural information for the glmS ribozyme. a) Secondary structure model of the glmS ribozyme from B. cereus and b) the equilibrium of β-anomer (1a), acyclic form (1b), and α-anomer (1c) of GlcN6P, the ligand of glmS ribozyme. The secondary structure model was adapted from a model for the glmS ribozyme from Thermoanaerobacter tengcongensis based on x-ray data.

FIG. 2 shows GlcN6P analogs and their influence on glmS ribozyme self-cleavage. a) Chemical structures of GlcN6P analogs. Differences from GlcN6P can be seen by comparing the structures to GlcN6P. b) Yields of ribozyme cleavage assays using a 200-nucleotide glmS RNA construct incubated with GlcN6P (1) or with various compounds as indicated. 5′ ³²P-labeled precursor (Pre) RNAs were incubated for 30 minutes in the presence of 1 mM effector as noted for each lane. Bar designated (-) reveals the extent of RNA cleavage when a reaction containing 1 mM GlcN6P was terminated with loading buffer at time zero. Data for all assays was corrected for the amount of cleaved RNA present in the reaction generating the lowest amount of cleavage (reaction containing 7). Open bars designate active compounds that were further examined to establish ribozyme rate constants. c) Observed rate constants (k_{obs}) for ribozyme cleavage and the ratio of rate constants (kₗ/k) measured using GlcN6P (1) versus the most active GlcN6P analogs, each at 100 μM. Rate constants for the remaining compounds are estimated to be less than 0.001 min⁻¹.

FIG. 3 shows observed rate constants for ribozyme self-cleavage at different concentrations of 8. Inset depicts three representative assays wherein radiolabeled precursor (Pre) are incubated for various times with different concentrations (c) of 8 as indicated. Cleaved (Clv) RNAs are separated by polyacrylamide gel electrophoresis (PAGE). Aliquots of ribozyme reactions were removed and terminated at 0, 4, 15.3 and 19.5 hours.

FIG. 4 shows predicted molecular recognition determinants of glmS ribozymes. Confirmation of the precise type or number of molecular contacts for some functional groups requires testing of additional analogs.

FIG. 5 shows concentration-dependent activation of the 200-nucleotide glmS ribozyme of B. cereus by GlcN6P analogs. Data for compounds 13 (filled squares), 9 (open squares), 8 (open triangles), 12 (open circles), and 4 (filled triangles) are depicted. The data for 8 is also presented in FIG. 3 of the main text.

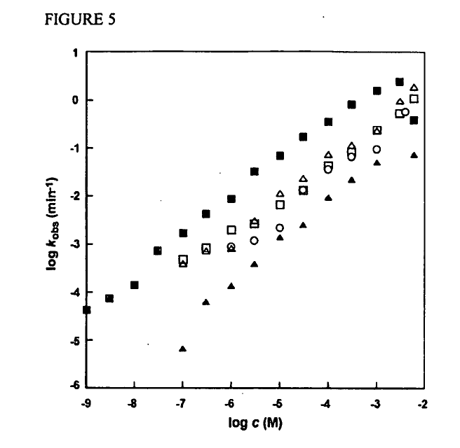

FIG. 6 shows the three dimensional crystal structure of the glmS riboswitch, the control of which is essential to bacterial cell wall biosynthesis and viability. The structure of the riboswitch shown is that of Bacillus anthracis. The structure reveals the three dimensional arrangement of the RNA in this ribozyme/riboswitch and shows the small molecular effector, glucosamine-6-phosphate, bound in the RNA's active site.

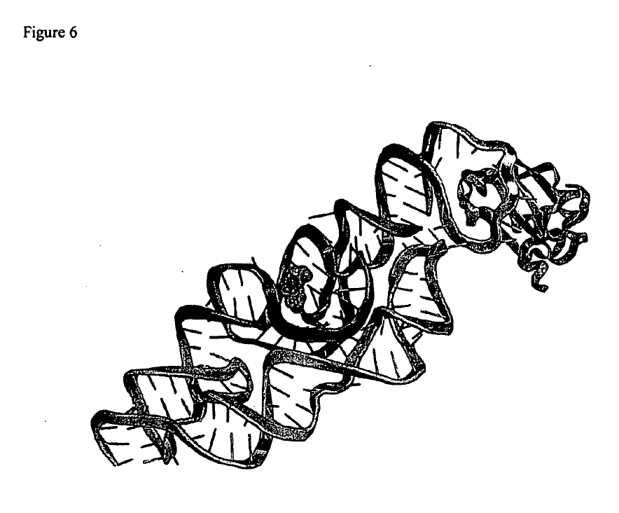

FIGS. 7A and 7B show the sequences and structures of two glmS ribozymes. A. A unimolecular glmS ribozyme from B. subtilis (from 5′ end to 3′ end, SEQ ID NOs:1-8). Arrow identifies the site of ribozyme-mediate cleavage stimulated by GlcN6P (Wolfson, Chem Biol 2006; 13: 1-3). B. A bimolecular glmS ribozyme construct derived from S. aureus (from 5′ end to 3′ end, SEQ ID NOs:9-18. This construct differs from the wild-type glmS ribozyme due to truncation of the P1 stem and the use of a 15-nucleotide substrate RNA (shown in grey; SEQ ID NOs:9 and 10). The substrate is labeled with a Cy3™ acceptor at its 5′-terminus and a 5/6-FAM donor at its 3′-terminus.

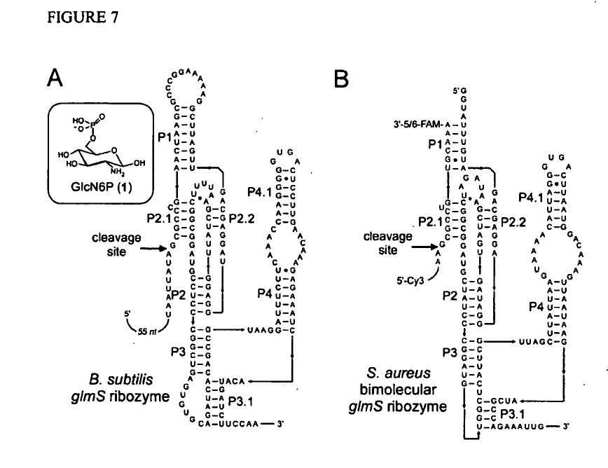

FIG. 8 shows the consensus sequence and structure for glmS ribozymes. Arrowhead identifies the site of cleavage. Circled nucleotides are conserved in at least 97% of glmS ribozyme representatives.

FIGS. 9A, 9B, and 9C show GlcN6P binding by the Bacillus anthracis glmS ribozyome. (A) Phosphate coordination by two magnesium ions in the GlmS ribozyme. Nucleotides in P2.1, A28, and G1 use water-mediated contacts to organize two hydrated magnesium ions and orient the GlcN6P phosphate oxygens in the active site. (B) Recognition of the GlcN6P sugar ring by nucleobase functional groups. The sugar contacts nucleotides A-42, U43 and G57, and the sugar and 3′-phosphate of G1. The quinine base at G1, which stacks on top of GlcN6P (FIG. 9A), is not shown to allow the hydrogen bonding contacts to be visualized. (C) Active site interations expected to stabilize this conformation are depicted as thin dashed lines. The catalytically critical interactions between the ethanolamine moiety of GlcN6P and the reactive phosphate are shown as thicker dashed lines. The scissile phosphate, 5′-O leaving group (which has been methylated), and 2′-O nucleophile are shown as spheres. The nucleotides are identified by numbering used for the glms riboswitch in Cochrane 2007, which is hereby incorporated by reference for this numbering.

### DETAILED DESCRIPTION OF THE INVENTION

The disclosed methods and compositions can be understood more readily by reference to the following detailed description of particular embodiments and the Examples included therein and to the Figures and their previous and following description.

Messenger RNAs are typically thought of as passive carriers of genetic information that , are acted upon by protein- or small RNA-regulatory factors and by ribosomes during the process of translation. It was discovered that certain mRNAs carry natural aptamer domains and that binding of specific metabolites directly to these RNA domains leads to modulation of gene expression. Natural riboswitches exhibit two surprising functions that are not typically associated with natural RNAs. First, the mRNA element can adopt distinct structural states wherein one structure serves as a precise binding pocket for its target metabolite. Second, the metabolite-induced allosteric interconversion between structural states causes a change in the level of gene expression by one of several distinct mechanisms. Riboswitches typically can be dissected into two separate domains: one that selectively binds the target (aptamer domain) and another that influences genetic control (expression platform). It is the dynamic interplay between these two domains that results in metabolite-dependent allosteric control of gene expression.

Distinct classes of riboswitches have been identified and are shown to selectively recognize activating compounds (referred to herein as trigger molecules). For example, coenzyme B₁₂, glycine, thiamine pyrophosphate (TPP), and flavin mononucleotide (FMN) activate riboswitches present in genes encoding key enzymes in metabolic or transport pathways of these compounds. The aptamer domain of each riboswitch class conforms to a highly conserved consensus sequence and structure. Thus, sequence homology searches can be used to identify related riboswitch domains. Riboswitch domains have been discovered in various organisms from bacteria, archaea, and eukarya.

The glmS ribozyme is a cis-cleaving catalytic riboswitch located in the 5′-UTR of bacterial mRNA that codes for glucosamine-6-phosphate synthetase (Winkler 2004). The ribozyme can be specifically activated for glmS-mRNA cleavage by the metabolite glucosamine-6-phosphate (GlcN6P), that is, the metabolic product of the glmS-encoded protein itself. This regulation thus relies on a feedback-inhibition mechanism that senses the presence of metabolites that serve as cell-wall precursors (Mayer and Famulok, ChemBioChem 2006, Vol. 7, p. 602-604, hereby incorporated by reference in its entirety for its teaching concerning glmS riboswitches).

### A. General Organization of Riboswitch RNAs

Bacterial riboswitch RNAs are genetic control elements that are located primarily within the 5′-untranslated region (5′-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). These conclusions are drawn from the observation that aptamer domains synthesized in vitro bind the appropriate ligand in the absence of the expression platform (see Examples 2, 3 and 6 of U.S. Application Publication No. 2005-0053951). Moreover, structural probing investigations suggest that the aptamer domain of most riboswitches adopts a particular secondary- and tertiary-structure fold when examined independently, that is essentially identical to the aptamer structure when examined in the context of the entire 5′ leader RNA. This indicates that, in many cases, the aptamer domain is a modular unit that folds independently of the expression platform (see Examples 2, 3 and 6 of U.S. Application Publication No. 2005-0053951).

Ultimately, the ligand-bound or unbound status of the aptamer domain is interpreted through the expression platform, which is responsible for exerting an influence upon gene expression. The view of a riboswitch as a modular element is further supported by the fact that aptamer domains are highly conserved amongst various organisms (and even between kingdoms as is observed for the TPP riboswitch), (N. Sudarsan, et al., RNA 2003, 9, 644) whereas the expression platform varies in sequence, structure, and in the mechanism by which expression of the appended open reading frame is controlled. For example, ligand binding to the TPP riboswitch of the tenA mRNA of B. subtilis causes transcription termination (A. S. Mironov, et al., Cell 2002, 111, 747). This expression platform is distinct in sequence and structure compared to the expression platform of the TPP riboswitch in the thiM mRNA from E. coli, wherein TPP binding causes inhibition of translation by a SD blocking mechanism (see Example 2 of U.S. Application Publication No. 2005-0053951). The TPP aptamer domain is easily recognizable and of near identical functional character between these two transcriptional units, but the genetic control mechanisms and the expression platforms that carry them out are very different.

Aptamer domains for riboswitch RNAs typically range from ˜70 to 170 nt in length (FIG. 11 of U.S. Application Publication No. 2005-0053951). This observation was somewhat unexpected given that in vitro evolution experiments identified a wide variety of small molecule-binding aptamers, which are considerably shorter in length and structural intricacy (T. Hermann, D. J. Patel, Science 2000, 287, 820; L. Gold, et al., Annual Review of Biochemistry 1995, 64, 763; M. Famulok, Current Opinion in Structural Biology 1999, 9, 324). Although the reasons for the substantial increase in complexity and information content of the natural aptamer sequences relative to artificial aptamers remains to be proven, this complexity is believed required to form RNA receptors that function with high affinity and selectivity. Apparent K_{D} values for the ligand-riboswitch complexes range from low nanomolar to low micromolar. It is also worth noting that some aptamer domains, when isolated from the appended expression platform, exhibit improved affinity for the target ligand over that of the intact riboswitch. (˜10 to 100-fold) (see Example 2 of U.S. Application Publication No. 2005-0053951). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

### B. Riboswitch Regulation of Transcription Termination in Bacteria

Bacteria primarily make use of two methods for termination of transcription. Certain genes incorporate a termination signal that is dependent upon the Rho protein, (J. P. Richardson, Biochimica et Biophysica Acta 2002, 1577, 251). while others make use of Rho-independent terminators (intrinsic terminators) to destabilize the transcription elongation complex (I. Gusarov, E. Nudler, Molecular Cell 1999, 3, 495; E. Nudler, M. E. Gottesman, Genes to Cells 2002, 7, 755). The latter RNA elements are composed of a GC-rich stem-loop followed by a stretch of 6-9 uridyl residues. Intrinsic terminators are widespread throughout bacterial genomes (F. Lillo, et al., 2002, 18, 971), and are typically located at the 3′-termini of genes or operons. Interestingly, an increasing number of examples are being observed for intrinsic terminators located within 5′-UTRs.

Amongst the wide variety of genetic regulatory strategies employed by bacteria there is a growing class of examples wherein RNA polymerase responds to a termination signal within the 5′-UTR in a regulated fashion (T. M. Henkin, Current Opinion in Microbiology 2000, 3, 149). During certain conditions the RNA polymerase complex is directed by external signals either to perceive or to ignore the termination signal. Although transcription initiation might occur without regulation, control over mRNA synthesis (and of gene expression) is ultimately dictated by regulation of the intrinsic terminator. Presumably, one of at least two mutually exclusive mRNA conformations results in the formation or disruption of the RNA structure that signals transcription termination. A trans-acting factor, which in some instances is a RNA (F. J. Grundy, et al., Proceedings of the National Academy of Sciences of the United States of America 2002, 99, 11121; T. M. Henkin, C. Yanofsky, Bioessays 2002, 24, 700) and in others is a protein (J. Stulke, Archives of Microbiology 2002, 177, 433), is generally required for receiving a particular intracellular signal and subsequently stabilizing one of the RNA conformations. Riboswitches offer a direct link between RNA structure modulation and the metabolite signals that are interpreted by the genetic control machinery.

The glmS ribozyme (Winkler 2004; Barrick 2004; McCarthy 2005; Wilkinson 2005; Soukup 2006; Roth 2006; Jansen 2006) from Bacillus cereus is a representative of a unique riboswitch (Mandal 2004; Winkler 2005) class whose members undergo self-cleavage with accelerated rate constants when bound to glucosamine-6-phosphate (GlcN6P). These metabolite-sensing ribozymes are found in numerous Gram-positive bacteria where they control expression of the glmS gene. The glmS gene product (glutamine-fructose-6-phosphate amidotransferase) generates GlcN6P (Badet-Denisot 1993; Milewski 2002) which binds to the ribozyme and triggers self-cleavage by internal phosphoester transfer (Winkler 2004). The ribozyme is embedded within the 5′ untranslated region (UTR) of the glmS messenger RNA and self-cleavage prevents GlmS protein production, thereby decreasing the concentration of GlcN6P. The combination of molecular sensing, self-cleavage, and gene control functions allows this small RNA to operate both as a ribozyme and as a riboswitch.

It has been shown that the glmS ribozyme from B. cereus, and the homologous ribozyme from Bacillus subtilis respond to GlcN6P with an apparent dissociation constant (K_{D}) of ˜200 μM (Winkler 2004; McCarthy 2005; Roth 2006). Although this K_{D} value is greater than those determined for most other natural riboswitches, glmS ribozymes exhibit a high level of molecular recognition specificity. For example, glucosamine-6-sulphate can induce ribozyme activation to the same extent as GlcN6P, albeit when present at concentrations that are ˜100-fold greater. In contrast, glucose-6-phosphate, wherein the 2-amine group of GlcN6P is replaced with a hydroxyl group, completely fails to trigger ribozyme action (Winkler 2004; McCarthy 2005).

Riboswitches must be capable of discriminating against compounds related to their natural ligands to prevent undesirable regulation of metabolic genes. However, it is possible to generate analogs that trigger riboswitch function and inhibit bacterial growth, as has been demonstrated for riboswitches that normally respond to lysine (Sudarsan 2003) and thiamine pyrophosphate (Sudarsan 2006). Proper expression of the GlmS protein is critical for bacterial viability (Badet-Denisot 1993; Milewski 2002), and analogs of GlcN6P that could interfere with normal gene expression by triggering glmS ribozyme activity might serve as new types of antimicrobial agents. Therefore, an increased understanding of the molecular recognition characteristics of glmS ribozymes was sought.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference to each of various individual and collective combinations and permutation of these compounds can not be explicitly disclosed, each is specifically contemplated and described herein. For example, if a riboswitch or aptamer domain is disclosed and discussed and a number of modifications that can be made to a number of molecules including the riboswitch or aptamer domain are discussed, each and every combination and permutation of riboswitch or aptamer domain and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Riboswitches

Riboswitches are expression control elements that are part of an RNA molecule to be expressed and that change state when bound by a trigger molecule. Riboswitches typically can be dissected into two separate domains: one that selectively binds the target (aptamer domain) and another that influences genetic control (expression platform domain). It is the dynamic interplay between these two domains that results in metabolite-dependent allosteric control of gene expression. Disclosed are isolated and recombinant riboswitches, recombinant constructs containing such riboswitches, heterologous sequences operably linked to such riboswitches, and cells and transgenic organisms harboring such riboswitches, riboswitch recombinant constructs, and riboswitches operably linked to heterologous sequences. The heterologous sequences can be, for example, sequences encoding proteins or peptides of interest, including reporter proteins or peptides. Preferred riboswitches are, or are derived from, naturally occurring riboswitches.

The disclosed riboswitches, including the derivatives and recombinant forms thereof, generally can be from any source, including naturally occurring riboswitches and riboswitches designed de novo. Any such riboswitches can be used in or with the disclosed methods. However, different types of riboswitches can be defined and some such sub-types can be useful in or with particular methods (generally as described elsewhere herein). Types of riboswitches include, for example, naturally occurring riboswitches, derivatives and modified forms of naturally occurring riboswitches, chimeric riboswitches, and recombinant riboswitches. A naturally occurring riboswitch is a riboswitch having the sequence of a riboswitch as found in nature. Such a naturally occurring riboswitch can be an isolated or recombinant form of the naturally occurring riboswitch as it occurs in nature. That is, the riboswitch has the same primary structure but has been isolated or engineered in a new genetic or nucleic acid context. Chimeric riboswitches can be made up of, for example, part of a riboswitch of any or of a particular class or type of riboswitch and part of a different riboswitch of the same or of any different class or type of riboswitch; part of a riboswitch of any or of a particular class or type of riboswitch and any non-riboswitch sequence or component. Recombinant riboswitches are riboswitches that have been isolated or engineered in a new genetic or nucleic acid context.

Riboswitches can have single or multiple aptamer domains. Aptamer domains in riboswitches having multiple aptamer domains can exhibit cooperative binding of trigger molecules or can not exhibit cooperative binding of trigger molecules (that is, the aptamers need not exhibit cooperative binding). In the latter case, the aptamer domains can be said to be independent binders. Riboswitches having multiple aptamers can have one or multiple expression platform domains. For example, a riboswitch having two aptamer domains that exhibit cooperative binding of their trigger molecules can be linked to a single expression platform domain that is regulated by both aptamer domains. Riboswitches having multiple aptamers can have one or more of the aptamers joined via a linker. Where such aptamers exhibit cooperative binding of trigger molecules, the linker can be a cooperative linker.

Aptamer domains can be said to exhibit cooperative binding if they have a Hill coefficient n between x and x-1, where x is the number of aptamer domains (or the number of binding sites on the aptamer domains) that are being analyzed for cooperative binding. Thus, for example, a riboswitch having two aptamer domains (such as glycine-responsive riboswitches) can be said to exhibit cooperative binding if the riboswitch has Hill coefficient between 2 and 1. It should be understood that the value of x used depends on the number of aptamer domains being analyzed for cooperative binding, not necessarily the number of aptamer domains present in the riboswitch. This makes sense because a riboswitch can have multiple aptamer domains where only some exhibit cooperative binding.

Disclosed are chimeric riboswitches containing heterologous aptamer domains and expression platform domains. That is, chimeric riboswitches are made up an aptamer domain from one source and an expression platform domain from another source. The heterologous sources can be from, for example, different specific riboswitches, different types of riboswitches, or different classes of riboswitches. The heterologous aptamers can also come from non-riboswitch aptamers. The heterologous expression platform domains can also come from non-riboswitch sources.

Modified or derivative riboswitches can be produced using in vitro selection and evolution techniques. In general, in vitro evolution techniques as applied to riboswitches involve producing a set of variant riboswitches where part(s) of the riboswitch sequence is varied while other parts of the riboswitch are held constant. Activation, deactivation or blocking (or other functional or structural criteria) of the set of variant riboswitches can then be assessed and those variant riboswitches meeting the criteria of interest are selected for use or further rounds of evolution. Useful base riboswitches for generation of variants are the specific and consensus riboswitches disclosed herein. Consensus riboswitches can be used to inform which part(s) of a riboswitch to vary for in vitro selection and evolution.

Also disclosed are modified riboswitches with altered regulation. The regulation of a riboswitch can be altered by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are inactivated riboswitches. Riboswitches can be inactivated by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch. Biosensor riboswitches can be used in various situations and platforms. For example, biosensor riboswitches can be used with solid supports, such as plates, chips, strips and wells.

Also disclosed are modified or derivative riboswitches that recognize new trigger molecules. New riboswitches and/or new aptamers that recognize new trigger molecules can be selected for, designed or derived from known riboswitches. This can be accomplished by, for example, producing a set of aptamer variants in a riboswitch, assessing the activation of the variant riboswitches in the presence of a compound of interest, selecting variant riboswitches that were activated (or, for example, the riboswitches that were the most highly or the most selectively activated), and repeating these steps until a variant riboswitch of a desired activity, specificity, combination of activity and specificity, or other combination of properties results.

In general, any aptamer domain can be adapted for use with any expression platform domain by designing or adapting a regulated strand in the expression platform domain to be complementary to the control strand of the aptamer domain. Alternatively, the sequence of the aptamer and control strands of an aptamer domain can be adapted so that the control strand is complementary to a functionally significant sequence in an expression platform. For example, the control strand can be adapted to be complementary to the Shine-Dalgarno sequence of an RNA such that, upon formation of a stem structure between the control strand and the SD sequence, the SD sequence becomes inaccessible to ribosomes, thus reducing or preventing translation initiation. Note that the aptamer strand would have corresponding changes in sequence to allow formation of a P1 stem in the aptamer domain. In the case of riboswitches having multiple aptamers exhibiting cooperative binding, one the P1 stem of the activating aptamer (the aptamer that interacts with the expression platform domain) need be designed to form a stem structure with the SD sequence.

As another example, a transcription terminator can be added to an RNA molecule (most conveniently in an untranslated region of the RNA) where part of the sequence of the transcription terminator is complementary to the control strand of an aptamer domain (the sequence will be the regulated strand). This will allow the control sequence of the aptamer domain to form alternative stem structures with the aptamer strand and the regulated strand, thus either forming or disrupting a transcription terminator stem upon activation or deactivation of the riboswitch. Any other expression element can be brought under the control of a riboswitch by similar design of alternative stem structures.

For transcription terminators controlled by riboswitches, the speed of transcription and spacing of the riboswitch and expression platform elements can be important for proper control. Transcription speed can be adjusted by, for example, including polymerase pausing elements (e.g., a series of uridine residues) to pause transcription and allow the riboswitch to form and sense trigger molecules.

Disclosed are regulatable gene expression constructs comprising a nucleic acid molecule encoding an RNA comprising a riboswitch operably linked to a coding region, wherein the riboswitch regulates expression of the RNA, wherein the riboswitch and coding region are heterologous. The riboswitch can comprise an aptamer domain and an expression platform domain, wherein the aptamer domain and the expression platform domain are heterologous. The riboswitch can comprise an aptamer domain and an expression platform domain, wherein the aptamer domain comprises a P1 stem, wherein the P1 stem comprises an aptamer strand and a control strand, wherein the expression platform domain comprises a regulated strand, wherein the regulated strand, the control strand, or both have been designed to form a stem structure. The riboswitch can comprise two or more aptamer domains and an expression platform domain, wherein at least one of the aptamer domains and the expression platform domain are heterologous. The riboswitch can comprise two or more aptamer domains and an expression platform domain, wherein at least one of the aptamer domains comprises a P1 stem, wherein the P1 stem comprises an aptamer strand and a control strand, wherein the expression platform domain comprises a regulated strand, wherein the regulated strand, the control strand, or both have been designed to form a stem structure.

1. Aptamer Domains

Aptamers are nucleic acid segments and structures that can bind selectively to particular compounds and classes of compounds. Riboswitches have aptamer domains that, upon binding of a trigger molecule result in a change in the state or structure of the riboswitch. In functional riboswitches, the state or structure of the expression platform domain linked to the aptamer domain changes when the trigger molecule binds to the aptamer domain. Aptamer domains of riboswitches can be derived from any source, including, for example, natural aptamer domains of riboswitches, artificial aptamers, engineered, selected, evolved or derived aptamers or aptamer domains. Aptamers in riboswitches generally have at least one portion that can interact, such as by forming a stem structure, with a portion of the linked expression platform domain. This stem structure will either form or be disrupted upon binding of the trigger molecule.

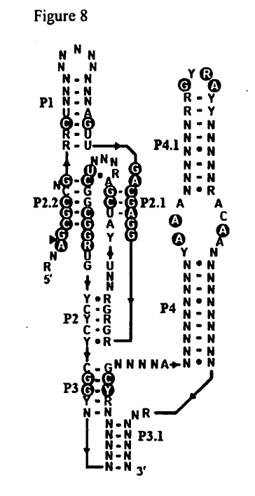

Consensus aptamer domains of a variety of natural riboswitches are shown in FIG. 11 of U.S. Application Publication No. 2005-0053951 and elsewhere herein. The consensus sequence and structure for the glmS ribozyme can be found in FIG. 8. These aptamer domains (including all of the direct variants embodied therein) can be used in riboswitches. The consensus sequences and structures indicate variations in sequence and structure. Aptamer domains that are within the indicated variations are referred to herein as direct variants. These aptamer domains can be modified to produce modified or variant aptamer domains. Conservative modifications include any change in base paired nucleotides such that the nucleotides in the pair remain complementary. Moderate modifications include changes in the length of stems or of loops (for which a length or length range is indicated) of less than or equal to 20% of the length range indicated. Loop and stem lengths are considered to be “indicated” where the consensus structure shows a stem or loop of a particular length or where a range of lengths is listed or depicted. Moderate modifications include changes in the length of stems or of loops (for which a length or length range is not indicated) of less than or equal to 40% of the length range indicated. Moderate modifications also include and functional variants of unspecified portions of the aptamer domain.

The P1 stem and its constituent strands can be modified in adapting aptamer domains for use with expression platforms and RNA molecules. Such modifications, which can be extensive, are referred to herein as P1 modifications. P1 modifications include changes to the sequence and/or length of the P1 stem of an aptamer domain. The aptamer domain shown in FIG. 8 is particularly useful as initial sequences for producing derived aptamer domains via in vitro selection or in vitro evolution techniques.

Aptamer domains of the disclosed riboswitches can also be used for any other purpose, and in any other context, as aptamers. For example, aptamers can be used to control ribozymes, other molecular switches, and any RNA molecule where a change in structure can affect function of the RNA.

2. Expression Platform Domains

Expression platform domains are a part of riboswitches that affect expression of the RNA molecule that contains the riboswitch. Expression platform domains generally have at least one portion that can interact, such as by forming a stem structure, with a portion of the linked aptamer domain. This stem structure will either form or be disrupted upon binding of the trigger molecule. The stem structure generally either is, or prevents formation of, an expression regulatory structure. An expression regulatory structure is a structure that allows, prevents, enhances or inhibits expression of an RNA molecule containing the structure. Examples include Shine-Dalgarno sequences, initiation codons, transcription terminators, and stability and processing signals.

### B. Trigger Molecules

Trigger molecules are molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques).

### C. Compounds

Also disclosed are compounds, and compositions containing such compounds, that can activate, deactivate or block a riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. A riboswitch can be blocked by, for example, binding of an analog of the trigger molecule that does not activate the riboswitch.

Also disclosed are compounds for altering expression of an RNA molecule, or of a gene encoding an RNA molecule, where the RNA molecule includes a riboswitch. This can be accomplished by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are compounds for regulating expression of an RNA molecule, or of a gene encoding an RNA molecule. Also disclosed are compounds for regulating expression of a naturally occurring gene or RNA that contains a riboswitch by activating, deactivating or blocking the riboswitch. If the gene is essential for survival of a cell or organism that harbors it, activating, deactivating or blocking the riboswitch can in death, stasis or debilitation of the cell or organism.

Also disclosed are compounds for regulating expression of an isolated, engineered or recombinant gene or RNA that contains a riboswitch by activating, deactivating or blocking the riboswitch. If the gene encodes a desired expression product, activating or deactivating the riboswitch can be used to induce expression of the gene and thus result in production of the expression product. If the gene encodes an inducer or repressor of gene expression or of another cellular process, activation, deactivation or blocking of the riboswitch can result in induction, repression, or de-repression of other, regulated genes or cellular processes. Many such secondary regulatory effects are known and can be adapted for use with riboswitches. An advantage of riboswitches as the primary control for such regulation is that riboswitch trigger molecules can be small, non-antigenic molecules.

Also disclosed are methods of identifying compounds that activate, deactivate or block a riboswitch. For example, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

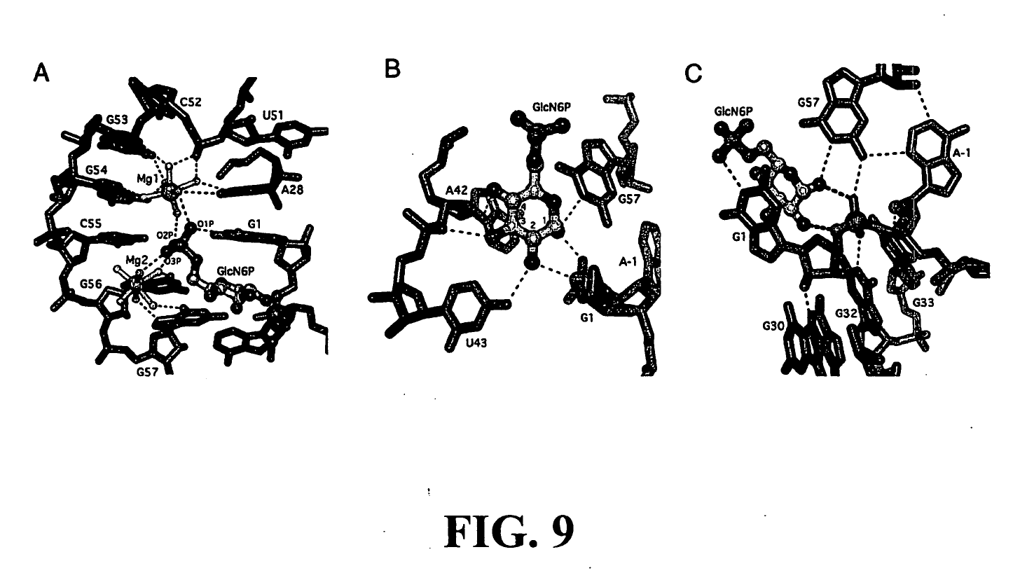

Compounds can also be identified using the atomic crystalline structure of a riboswitch. The atomic coordinates of the atomic structure of the glmS riboswitch are listed in Table 2. The atomic structure of the active site and binding pocket as depicted in FIG. 9 and the atomic coordinates of the active site and binding pocket depicted in FIG. 9 contained within Table 2 can also be used. Compounds can be identified using the crystalline structure of a riboswitch by, for example, modeling the atomic structure of the riboswitch with a test compound; and determining if the test compound interacts with the riboswitch. This can be done by using a predicted minimum interaction energy, a predicted bind constant, a predicted dissociation constant, or a combination, for the test compound in the model of the riboswitch. Compounds can also be identified by, for example, asessing the fit between the riboswtich and a compound known to bind the riboswitch (such as the trigger molecule), identify sites where the compound can be changed with little or no obvious adverse effects on binding of the compound, and incorporating one or more such alterations to produce a new compound. The method of identifying compounds that interact with a riboswitch can also involve production of the compounds so identified.

Typically the method first utilizes a 3-dimensional structure of the riboswitch with a compound, also referred to as a “known compound” or “known target”. Any of the trigger molecules and compounds disclosed herein can be used as such a known compound. The structure of the riboswitch can be determined using any known means, such as crystallography or solution NMR spectroscopy. That structure can also be obtained through computer molecular modeling simulation programs, such as AutoDock. The methods can involve determining the amount of binding, such as determining the binding energy, between a riboswitch, and a potential compound for that riboswitch. An active compound is a compound that has some activity against a riboswitch, such as inhibiting the riboswitch's activity or enhancing the riboswitch's activity. In addition, the potential compound can be an analog, which has some structural relationship to a known compound for the molecule. Any of the trigger molecules, known compounds, and compounds disclosed herein can be used as the basis of or to derive a potential compound.

The identity or relationship of the structure, properties, interaction or binding parameters, and the like of the known compound and potential compound can be viewed in number of ways. For example, any of the measures or interaction parameters that can be measured or assessed using the structural model, and such measures and parameters obtained for a known compound and a potential compound can be compared. One can look at the identity between the entire known compound and the potential compound. One can also look at the identity between the potential compound, such as an analog, and the know compound only in the domain where the potential compound interacts with the riboswitch. One can also look at the identity between the potential compound and the known compound at the level of a sub-domain, such as only those moieties or atoms in the potential compound which are within 7 Å, 6 Å, 5 Å, 4 Å, 3Å, or 2 Å of a moiety or atom which is in contact with the riboswitch in the known compound. Generally, the more specific the sub-domain the higher the identity will be between the moieties of the potential compound and the known compound. For example, there can be 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater identity between the known compound and potential compound as a whole, 50% or greater, 55% or greater, 60% or greater, 65% or greater, 70% or greater, 75% or greater, 80% or greater, 85% or greater, 90% or greater, 95% or greater identity between the binding domain of the known compound and the potential compound, and 70% or greater, 75% or greater, 80% or greater, 85% or greater, 90% or greater, 95% or greater identity between the moieties or atoms of the potential compound that correspond to the moieties or atoms of the known compound which are within 5 Å of a moiety or atom which interacts with the riboswitch. Another sub-domain is a sub-domain of moieties or atoms which actually contact the riboswitch. In this case the identity can be, for example, greater than 50%, 55%, 60%, 65%, 70%, 75%, 80%, 85%, 90%, 95% or higher.

Typically, the potential compounds exist in a family of potential compounds, i.e. a set of analogs, all of which have some structural relationship to the known compound for the riboswitch. A family consisting of any number of members can be screened. The maximum number of members in the family is only limited by the amount of computer power available to screen each member in a desired amount of time. The methods can involve at least one template structure of the riboswitch and a target, often this would be with a known target. It is not required that this structure be existent, as it can be generated, in some cases during the disclosed methods, using standard structure determination techniques. It is preferred that a real structure exist at the time the methods are employed.

The methods can also involve modeling the structure of the potential compound, using information from the structure of the known compound. This modeling can be performed in any way, and as described herein.

The conformation and position of the potential compound can be held fixed during the calculations; that is, it can be assumed that the riboswitch binds in exactly the same orientation to the potential compound as it does to a known compound.

Then, a binding energy (or other property or parameter) can be determined between the riboswitch and the potential compound, and if the binding energy (or other property or parameter) meets certain criteria, then the potential compound can be designated as an actual compound, i.e. one that is likely to interact with the riboswitch. Although the following refers to the use of binding energy, it should be understood that any property or parameter involving the interaction or modeling of a compound and a riboswitch can be used. The criterion can be that the computed binding energy of the riboswitch with the potential compound is similar to, or more favorable than, the computed binding energy of the same riboswitch with a known compound. For example, an actual compound can be a compound where the computed binding energy as discussed herein is, for example, at least 60%, 65%, 70%, 75%, 80%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 120%, 130%, 140%, 150%, 200%, 250%, 300%, 350%, 400%, 450%, 500%, 600%, 700%, 800%, 900%, 1000%, or greater than that of the known compound binding energy. An actual compound can also be a compound which after ordering all potential compounds in terms of the strength of their binding energies, are the compounds which are in the top 20%, 19%, 18%, 17%, 16%, 15%, 14%, 13%, 12%, 11%, 10%, 9%, 8%, 7%, 6%, 5%, 4%, 3%, 2%, 1% of computed binding strengths, of for example, a set of potential compounds where the set is at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, 175, 200, 225, 250, 275, 300, 350, 400, 500, 700, or a 1000 potential compounds.

It is also understood that once a potential compound is identified, as disclosed herein, traditional testing and analysis can be performed, such as performing a biological assay using the riboswitch and the actual compound to further define the ability of the actual compound to interact with and/or modulate the riboswitch. The disclosed methods can include the step of assaying the activity of the riboswitch and compound, as well as performing, for example, combinatorial chemistry studies using libraries based on the riboswitch, for example.

Energy calculations can be based on, for example, molecular or quantum mechanics. Molecular mechanics approximates the energy of a system by summing a series of empirical functions representing components of the total energy like bond stretching, van der Waals forces, or electrostatic interactions. Quantum mechanics methods use various degrees of approximation to solve the Schrodinger equation. These methods deal with electronic structure, allowing for the characterization of chemical reactions.

Potential compounds of the riboswitch can be identified. This can be accomplished by selecting potential compounds with a given similarity to the known compound. For example, compounds in the same family as the known compound can be selected.

To prepare each riboswitch for calculation, atoms can be built in that were unresolved or absent from the crystal structures of the potential compound. This can be done, for example, using the PRODRG webserver http://www.davapc1.bioch.dundee.ac.uk./programs/prodrg, or standard molecular modeling programs such as InsightII, Quanta (both at www.accelrys.com), CNS (Brunger et al., Crystallography & NMR system: a new software suite for macromolecular structure determination. Acta Crystallogr. D 54, 905-921 (1998)), or any other molecular modeling system capable of preparing the riboswitch structure.

The binding energy (or other property or parameter) of the potential compound and riboswitch can then be calculated. There are numerous means for carrying this out. For example, the sampling of sidechain positions and the computation of the binding thermodynamics can be accomplished using an empirical function that models the energy of the potential compound-molecule as a sum of electrostatic and van der Waals interactions between all pairs of atoms within the model. Any other computational method for scoring the binding energy of the potential compound with the riboswitch can be used (H. Gohlke, & G. Klebe. Approaches to the description and prediction of the binding affinity of small-molecule ligands to macromolecular receptors. Angew. Chem. Int. Ed. 41, 2644-4676 (2002)). Examples of such scoring methods include, but are not limited to, those implemented in programs such as AutoDock (G. M. Morris et al. Automated docking using a Lamarckian genetic algorithm and an empirical binding free energy function. J. Comput. Chem. 19, 1639-1662 (1998)), Gold (G. Jones et al. Molecular recognition of receptor sites using a genetic algorithm with a description of desolvation. J. Mol. Biol. 245, 43-53 (1995)), Chem-Score (M. D. Eldridge et al. J. Comput.—Aided Mol. Des. 11, 425-445 (1997)) and Drug-Score (H. Gohlke et al. Knowledge-based scoring function to predict protein-ligand interactions. J. Mol. Biol. 295, 337-356 (2000)).

Rotamer libraries are known to those of skill in the art and can be obtained from a variety of sources, including the interne. Rotamers are low energy side-chain conformations. The use of a library of rotamers allows for the modeling of a structure to try the most likely side-chain conformations, saving time and producing a structure that is more likely to be correct. The use of a library of rotamers can be restricted to those residues that are within a given region of the potential compound, for example, at the binding site, or within a specified distance of the compound. The latter distance can be set at any desired length, for example, the potential compound can be 2, 3, 4, 5, 6, 7, 8, or 9 Å from any atom of the molecule.

Electrostatic interactions between every pair of atoms can be calculated, for example, using a Coulombic model with the formula:

E_{elec}=332.08 q₁ q₂/εr. where q₁ and q₂ are partial atomic charges, r is the distance between them, and ε is the dielectric constant.

Partial atomic charges can be taken from existing parameter sets that have been developed to describe charge distributions in molecules. Example parameter sets include, but are not limited to, PARSE (D. A. Sitkoff et al. Accurate calculation of hydration free-energies using macroscopic solvent models. J. Phys. Chem. 98, 1978-1988 (1994)), CHARMM (MacKerell et al. All-atom empirical potential for molecular modeling and dynamics studies of proteins. J. Phys. Chem. B 102, 3586-3616, 1998) and AMBER (W. D. Cornell et al. A 2^{nd} generation force-field for the simulation of proteins, nucleic-acids, and organic-molecules. J. Am. Chem. Soc. 117. 5179-5195 (1995)). Partial charges for atoms can be assigned either by analogy with those of similar functional groups, or by empirical assignment methods such as that implemented in the PRODRG server (D. M. F. van Aalten et al. PRODRG, a program for generating molecular topologies and unique molecular descriptors from coordinates of small molecules. J. Comput.—Aided Mol. Design 10, 255-262 (1996)), or by the use of standard quantum mechanical calculation methods (for example, C. I. Bayly et al. A well-behaved electrostatic potential based method using charge restraints for deriving atomic charges—the RESP model. J. Phys. Chem. 97, 10269-10280, (1993)).

The electrostatic interaction can also be calculated by more elaborate methodologies that incorporate electrostatic desolvation effects. These can include explicit solvent and implicit solvent models: in the former, water molecules are directly included in the calculations, whereas in the latter, the effects of water are described by a dielectric continuum approach. Specific examples of implicit solvent methods for calculating electrostatic interactions include but are not limited to: Poisson-Boltzmann based methods and Generalized Born methods (M. Feig & C. L. Brooks. Recent advances in the development and application of implicit solvent models in biomolecule simulations. Curr. Opin. Struct. Biol. 14, 217-224 (2004)).

van der Waals and hydrophobic interactions between pairs of atoms (where both atoms are either sulfur or carbon) can be calculated using a simple Lennard-Jones formalism with the following equation:

E_{vdw}ε={σₐₜₜ¹²/r¹²−σₐₜₜ⁶/r⁶}. where ε is an energy, r is the distance between the two atoms and σₐₜₜ is the distance at which the energy of interaction is zero.

van der Waals interactions between pairs of atoms (where one or both atoms are neither sulfur nor carbon) can be calculated using a simple repulsive energy term:

E_{vdw}=ε{σᵣₑₚ¹²/r¹²56 . where ε is an energy, r is the distance between the two atoms and σᵣₑₚ determines the distance at which the repulsive interaction is equal to ε.

Hydrophobic interactions between atoms can also be calculated using a variety of other methods known to those skilled in the art. For example, the energetic contribution can be calculated as being proportional to the amount of solvent accessible surface area of the ligand and receptor that is buried when the complex is formed. Such contributions can be expressed in terms of interactions between pairs of atoms, such as in the method proposed by Street & Mayo (A. G. Street & S. L. Mayo. Pairwise calculation of protein solvent-accessible surface areas. Folding & Design 3, 253-258 (1998)). Any other implementation of a formalism for describing hydrophobic or van der Waals or other energetic contributions can be included in the calculations.

Binding energies can be calculated for each potential compound-riboswitch interaction. For example, Monte Carlo sampling can be conducted in the presence and absence of the riboswitch, and the average energy in each simulation calculated. A binding energy for the riboswitch with the potential compound can then be calculated as the difference between the two calculated average energies.

The computed binding energy of a potential compound with the riboswitch can be compared with the computed binding energy of a known compound with the riboswitch to determine if the potential compound is likely to be an actual compound. These results can then be confirmed using experimental data, wherein the actual interaction between the riboswitch and compound can be measured. Examples of methods that can be used to determine an actual interaction between the riboswitch and the compound include but are not limited to: equilibrium dialysis measurements (wherein binding of a radioactive form of the compound to the riboswitch is detected), enzyme inhibition assays (wherein the activity of the riboswitch can be monitored in the presence and absence of the compound), and chemical shift perturbation measurements (wherein binding of the riboswitch to the potential compound is monitored by observing changes in NMR chemical shifts of atoms).

Modeling can be performed on or with the aid of a computer, a computer program, or a computer operating program. The computer can be made to display an image of the structure in 3D or represented as 3D. The image can be of any or all of the structure represented by the atomic coordinates of Table 2, for example, the structure represented by the atomic structure of the active site and binding pocket as depicted in FIG. 9 and the atomic coordinates of the active site and binding pocket depicted in FIG. 9 contained within Table 2 can be displayed. Any potion of the structure represented by the atomic coordinates of Table 2 that can be used to model and/or assess the ability of a compound to bind or interact specifically with a glmS riboswitch can be used for modeling and related methods as described herein.

After the atomic crystalline structure of the riboswitch has been modeled with a potential compound, further testing can be carried out to determine the actual interaction between the riboswitch and the compound. For example, multiple different approaches can be used to detect binding RNAs, including allosteric ribozyme assays using gel-based and chip-based detection methods, and in-line probing assays. High throughput testing can also be accomplished by using, for example, fluorescent detection methods. For example, the natural catalytic activity of a glucosamine-6-phosphate sensing riboswitch that controls gene expression by activating RNA-cleaving ribozyme can be used. This ribozyme can be reconfigured to cleave separate substrate molecules with multiple turnover kinetics. Therefore, a fluorescent group held in proximity to a quenching group can be uncoupled (and therefore become more fluorescent) if a compound triggers ribozyme function. Second, molecular beacon technology can be employed. This creates a system that suppresses fluorescence if a compound prevents the beacon from docking to the riboswitch RNA. Either approach can be applied to any of the riboswitch classes by using RNA engineering strategies described herein.

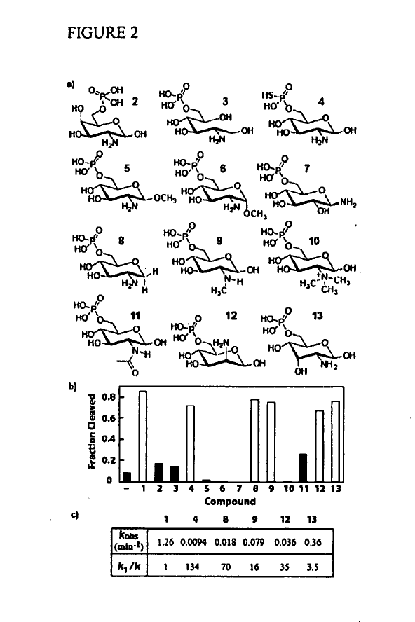

Also disclosed herein are analogs that interact with the glmS riboswitch disclosed herein. Examples of such analogs can be found in FIG. 2. Many of the compounds synthesized and tested bind the glmS riboswitch with constants that are equal to or better than that of GlcN6P. The fact that appendages with highly variable chemical composition exhibit function shows that numerous variations of these chemical scaffolds can be generated and tested for function in vitro and inside cells. Specifically, further modified versions of these compounds can have improved binding to the glmS riboswitch by making new contacts to other functional groups in the RNA structure. Furthermore, modulation of bioavailability, toxicity, and synthetic ease (among other characteristics) can be tunable by making modifications in these two regions of the scaffold, as the structural model for the riboswitch shows many modifications are possible at these sites.

High-throughput screening can also be used to reveal entirely new chemical scaffolds that also bind to riboswitch RNAs either with standard or non-standard modes of molecular recognition. Since riboswitches are the first major form of natural metabolite-binding RNAs to be discovered, there has been little effort made previously to create binding assays that can be adapted for high-throughput screening. Multiple different approaches can be used to detect metabolite binding RNAs, including allosteric ribozyme assays using gel-based and chip-based detection methods, and in-line probing assays. Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For the purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below. The term “lower alkyl” is an alkyl group with 6 or fewer carbon atoms, e.g., methyl, ethyl, propyl, isopropyl, butyl, sec-butyl, iso-butyl, tert-butyl, pentyl, hexyl, and the like.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “halogenated alkyl” is used in another, it is not meant to imply that the term “alkyl” does not also refer to specific terms such as “halogenated alkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bonded through a single, terminal ether linkage; that is, an “alkoxy” group can be defined as —OA¹ where A² is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “sulfo-oxo” as used herein is represented by the formulas —S(O)A¹ (i.e., “sulfonyl”), A¹ S(O)A² (i.e., “sulfoxide”), —S(O)₂A¹, A¹SO₂A² (i.e., “sulfone”),

—OS(O)₂A¹, or —OS(O)₂OA¹, where A₁ and A² can be hydrogen, an alkyl, halogenated alkyl, alkenyl, alkynyl, aryl, heteroaryl, cycloalkyl, cycloalkenyl, heterocycloalkyl, or heterocycloalkenyl group described above. Throughout this specification “S(O)” is a short hand notation for S═O.

The term “sulfonylamino” or “sulfonamide” as used herein is represented by the formula —S(O)₂NH—.

The term “thiol” as used herein is represented by the formula —SH.

As used herein, “Rⁿ” where n is some integer can independently possess one or more of the groups listed above. For example, if R¹⁰ contains an aryl group, one of the hydrogen atoms of the aryl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Compounds useful with glmS-responsive riboswitches (and riboswitches derived from glmS-responsive riboswitches) include compounds represented by Formula I:

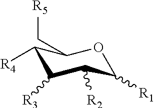

or pharmaceutically acceptable salts thereof, physiologically hydrolyzable and acceptable esters thereof, or both,

wherein R₁ is H, OH, SH, NH₂, or CH₃,

wherein R₂ is NH—R₆, wherein R₆ is H, CH₃, C₂H₅, n-propyl, C(O)CH₃, C(O)C₂H₅, C(O)n-propyl, C(O)iso-propyl, C(O)OCH₃, C(O)OC₂H₅, C(O)NH₂, or NH₂,

wherein R₃ is H, OH, SH, NH₂, or CH₃,

wherein R₄ is a hydrogen bond donor,

wherein R₅ is a hydrogen bond acceptor,

wherein the compound is not glucosamine-6-phosphate.

In one example, R₄ is OH, SH, NH₂, NH₃+, CH₂OH, CH(OH)CH₃, CH₂CH₂OH, CH₂SH, CH(SH)CH₃, CH₂CH₂SH, CH₂NH₂, CH(NH₂)CH₃, CH₂CH₂NH₃, CO₂H, CONH₂, CONHalkyl, ═NH, ═NOH, ═NSH, ═NCO₂H, ═CH₂, CH═NH, CH═NOH, CH═NSH, CH═NCO₂H, OCH₂OH, OCH₂CH₂OH, PhOH, NHalkyl, NHNH₂, NHNHalkyl, NHCOalkyl, NHCO₂alkyl, NHCONH₂, NHSO₂alkyl, or NHOalkyl.

In another example, R₄ is not OH when R₁ is H or OH and R₂ is NH₂ or NHCH₃.

In another example, R₅ can be OP(O)(OH)₂, OP(S)(OH)₂, OP(O)OHSH, OS(O)₂OH, or OS(O)₂SH. R₅ can also be OS(O)₂OH or OS(O)₂SH. R₅ can be negatively charged. R₅ can be ═O, CO₂R₉, OCO₂R₉, OCH₂OR₉, OC₂H₅OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, PO₂(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, wherein R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), —C(CH₃)₃, or —CF₃.

In another example, R₅ can also be ═O, OH, OR₉, COR₉, CN, NO₂, tetrazole, SOR₉, N(R₉)₂, CO₂R₉, OCO₂R₉, OCH₂OR₉, OC₂H₅OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, PO₂(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, wherein R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), —C(CH₃)₃, or —CF₃.

R₄ can be NH₂, NH₃⁺, OH, SH, NOH, NHNH₂, NHNH₃⁺, CO₂H, SO₂OH, B(OH)₂, or imidazolium.

R₄ can also be NH₂, NH₃⁺, SH, NOH, NHNH₂, NHNH₃⁺, CO₂H, SO₂OH, B(OH)₂, or imidazolium.

It is to be understood that while a particular moiety or group can be referred to herein as a hydrogen bond donor or acceptor, this terminology is used to merely categorize the various substituents for ease of reference. Such language should not be interpreted to mean that a particular moiety actually participates in hydrogen bonding with the riboswitch or some other compound. It is possible that, for example, a moiety referred to herein as a hydrogen bond acceptor (or donor) could solely or additionally be involved in hydrophobic, ionic, van de Waals, or other type of interaction with the riboswitch or other compound.

It is also understood that certain groups disclosed herein can be referred to herein as both a hydrogen bond acceptor and a hydrogen bond donor. For example, —OH can be a hydrogen bond donor by donating the hydrogen atom; —OH can also be a hydrogen bond acceptor through one or more of the nonbonded electron pairs on the oxygen atom. Thus, throughout the specification various moieties can be a hydrogen bond donor and acceptor and can be referred to as such.

Every compound within the above definition is intended to be and should be considered to be specifically disclosed herein. Further, every subgroup that can be identified within the above definition is intended to be and should be considered to be specifically disclosed herein. As a result, it is specifically contemplated that any compound, or subgroup of compounds can be either specifically included for or excluded from use or included in or excluded from a list of compounds. As an example, a group of compounds is contemplated where each compound is as defined above and is able to activate a glmS-responsive riboswitch.

It should be understood that particular contacts and interactions (such as hydrogen bond donation or acceptance) described herein for compounds interacting with riboswitches are preferred but are not essential for interaction of a compound with a riboswitch. For example, compounds can interact with riboswitches with less affinity and/or specificity than compounds having the disclosed contacts and interactions. Further, different or additional functional groups on the compounds can introduce new, different and/or compensating contacts with the riboswitches. For example, for glmS riboswitches, large functional groups can be used. Such functional groups can have, and can be designed to have, contacts and interactions with other part of the riboswitch. Such contacts and interactions can compensate for contacts and interactions of the trigger molecules and core structure.

### D. Constructs, Vectors and Expression Systems

The disclosed glmS riboswitches can be used with any suitable expression system. Recombinant expression is usefully accomplished using a vector, such as a plasmid. The vector can include a promoter operably linked to riboswitch-encoding sequence and RNA to be expression (e.g., RNA encoding a protein). The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying riboswitch-regulated constructs can be produced. Such expression vectors include, for example, pET, pET3d, pCR2.1, pBAD, pUC, and yeast vectors. The vectors can be used, for example, in a variety of in vivo and in vitro situation.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ (Laimins, 1981) or 3′ (Lusky et al., 1983) to the transcription unit. Furthermore, enhancers can be within an intron (Banerji et al., 1983) as well as within the coding sequence itself (Osborne et al., 1984). They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The vector can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein.

In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg,1982), mycophenolic acid, (Mulligan and Berg, 1980) or hygromycin (Sugden et al., 1985).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

1. Viral Vectors

Preferred viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Preferred retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

i. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

ii. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A preferred viral vector is one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

2. Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

It is preferred that the promoter and/or enhancer region be active in all eukaryotic cell types. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In a preferred embodiment of the transcription unit, the polyadenylation region is derived from the SV40 early polyadenylation signal and consists of about 400 bases. It is also preferred that the transcribed units contain other standard sequences alone or in combination with the above sequences improve expression from, or stability of, the construct.

3. Markers

The vectors can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### E. Biosensor Riboswitches

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, glmS biosensor riboswitches operably linked to a reporter RNA that encodes a protein that serves as or is involved in producing a signal can be used in vivo by engineering a cell or organism to harbor a nucleic acid construct encoding the glmS riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch, such as glmS.

### F. Reporter Proteins and Peptides

For assessing activation of a riboswitch, or for biosensor riboswitches, a reporter protein or peptide can be used. The reporter protein or peptide can be encoded by the RNA the expression of which is regulated by the riboswitch. The examples describe the use of some specific reporter proteins. The use of reporter proteins and peptides is well known and can be adapted easily for use with riboswitches. The reporter proteins can be any protein or peptide that can be detected or that produces a detectable signal. Preferably, the presence of the protein or peptide can be detected using standard techniques (e.g., radioimmunoassay, radio-labeling, immunoassay, assay for enzymatic activity, absorbance, fluorescence, luminescence, and Western blot). More preferably, the level of the reporter protein is easily quantifiable using standard techniques even at low levels. Useful reporter proteins include luciferases, green fluorescent proteins and their derivatives, such as firefly luciferase (FL) from Photinus pyralis, and Renilla luciferase (RL) from Renilla reniformis.

### G. Conformation Dependent Labels

Conformation dependent labels refer to all labels that produce a change in fluorescence intensity or wavelength based on a change in the form or conformation of the molecule or compound (such as a riboswitch) with which the label is associated. Examples of conformation dependent labels used in the context of probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Such labels, and, in particular, the principles of their function, can be adapted for use with riboswitches. Several types of conformation dependent labels are reviewed in Schweitzer and Kingsmore, Curr. Opin. Biotech. 12:21-27 (2001).

Stem quenched labels, a form of conformation dependent labels, are fluorescent labels positioned on a nucleic acid such that when a stem structure forms a quenching moiety is brought into proximity such that fluorescence from the label is quenched. When the stem is disrupted (such as when a riboswitch containing the label is activated), the quenching moiety is no longer in proximity to the fluorescent label and fluorescence increases. Examples of this effect can be found in molecular beacons, fluorescent triplex oligos, triplex molecular beacons, triplex FRET probes, and QPNA probes, the operational principles of which can be adapted for use with riboswitches.

Stem activated labels, a form of conformation dependent labels, are labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules (such as riboswitches) such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with riboswitches.

### H. Detection Labels

To aid in detection and quantitation of riboswitch activation, deactivation or blocking, or expression of nucleic acids or protein produced upon activation, deactivation or blocking of riboswitches, detection labels can be incorporated into detection probes or detection molecules or directly incorporated into expressed nucleic acids or proteins. As used herein, a detection label is any molecule that can be associated with nucleic acid or protein, directly or indirectly, and which results in a measurable, detectable signal, either directly or indirectly. Many such labels are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy Fl, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein(5-carboxyfluorescein-N-hydroxysuccinimide ester), rhodamine(5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a useful form of detection label for direct incorporation into expressed nucleic acids during synthesis. Examples of detection labels that can be incorporated into nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A useful nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′(5′-chloro)tricyclo[3.3.1.1^{3,7}]decane]-4-yl)phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, molecules and methods to label and detect activated or deactivated riboswitches or nucleic acid or protein produced in the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with a compound or composition to be detected and to which one or more detection labels are coupled.

### I. Sequence Similarities

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed riboswitches, aptamers, expression platforms, genes and proteins herein, is through defining the variants and derivatives in terms of homology to specific known sequences. This identity of particular sequences disclosed herein is also discussed elsewhere herein. In general, variants of riboswitches, aptamers, expression platforms, genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic'acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### J. Hybridization and Selective Hybridization

The term hybridization typically means a sequence driven interaction between at least two nucleic acid molecules, such as a primer or a probe and a riboswitch or a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid is in for example, 10 or 100 or 1000 fold excess. This type of assay can be performed at under conditions where both the limiting and non-limiting nucleic acids are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### K. Nucleic Acids

There are a variety of molecules disclosed herein that are nucleic acid based, including, for example, riboswitches, aptamers, and nucleic acids that encode riboswitches and aptamers. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, riboswitches, aptamers, expression platforms and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorportion into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH₂)nO]mCH₃, —O(CH₂)nOCH₃, —O(CH₂)nNH₂, —O(CH₂)nCH₃, —O(CH₂)n-ONH₂, and —O(CH₂)nON[(CH₂)nCH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs can also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides and nucleic acids can be referred to as chimeric oligonucleotides and chimeric nucleic acids.

### L. Solid Supports

Solid supports are solid-state substrates or supports with which molecules (such as trigger molecules) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds and/or probes can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### M. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting compounds, the kit comprising one or more biosensor riboswitches. The kits also can contain reagents and labels for detecting activation of the riboswitches.

### N. Mixtures

Disclosed are mixtures formed by performing or preparing to perform the disclosed method. For example, disclosed are mixtures comprising riboswitches and trigger molecules.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### O. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising biosensor riboswitches, a solid support and a signal-reading device.

### P. Data Structures and Computer Control

Disclosed are data structures used in, generated by, or generated from, the disclosed method. Data structures generally are any form of data, information, and/or objects collected, organized, stored, and/or embodied in a composition or medium. Riboswitch structures and activation measurements stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Methods

Disclosed are methods for activating, deactivating or blocking a riboswitch. Such methods can involve, for example, bringing into contact a riboswitch and a compound or trigger molecule that can activate, deactivate or block the riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. Thus, the disclosed method of deactivating a riboswitch can involve, for example, removing a trigger molecule (or other activating compound) from the presence or contact with the riboswitch. A riboswitch can be blocked by, for example, binding of an analog of the trigger molecule that does not activate the riboswitch.

Also disclosed are methods for altering expression of an RNA molecule, or of a gene encoding an RNA molecule, where the RNA molecule includes a riboswitch, by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent . expression of the RNA molecule or promote or increase expression of the RNA molecule.

Also disclosed are methods for regulating expression of a naturally occurring gene or RNA that contains a riboswitch by activating, deactivating or blocking the riboswitch. If the gene is essential for survival of a cell or organism that harbors it, activating, deactivating or blocking the riboswitch can result in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects. The compounds that have these antimicrobial effects are considered to be bacteriostatic or bacteriocidal.

Also disclosed are methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Also disclosed herein is a method of identifying a compound that interacts with a riboswitch comprising: modeling the atomic structure the riboswitch with a test compound; and determining if the test compound interacts with the riboswitch. Determining if the test compound interacts with the riboswitch can be accomplished by, for example, determining a predicted minimum interaction energy, a predicted bind constant, a predicted dissociation constant, or a combination, for the test compound in the model of the riboswitch, as described elsewhere herein. Determining if the test compound interacts with the riboswitch can be accomplished by, for example, determining one or more predicted bonds, one or more predicted interactions, or a combination, of the test compound with the model of the riboswitch. The predicted interactions can be selected from the group consisting of, for example, van der Waals interactions, hydrogen bonds, electrostatic interactions, hydrophobic interactions, or a combination, as described above. In one example, the riboswitch is a guanine riboswitch.

Atomic contacts can be determined when interaction with the riboswitch is determined, thereby determining the interaction of the test compound with the riboswitch. Analogs of the test compound can be identified, and it can be determined if the analogs of the test compound interact with the riboswitch.

Also disclosed are methods of killing or inhibiting bacteria, comprising contacting the bacteria with a compound disclosed herein or identified by the methods disclosed herein.

Also disclosed are methods of identifying compounds that activate, deactivate or block a riboswitch. For examples, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

In addition to the methods disclosed elsewhere herein, identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are methods of detecting compounds using biosensor riboswitches. The method can include bringing into contact a test sample and a biosensor riboswitch and assessing the activation of the biosensor riboswitch. Activation of the biosensor riboswitch indicates the presence of the trigger molecule for the biosensor riboswitch in the test sample. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, glmS biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a glmS riboswitch that includes a conformation dependent label, the signal from which changes depending on the activation state of the riboswitch. Such a biosensor riboswitch preferably uses an aptamer domain from or derived from a naturally occurring glmS riboswitch.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Disclosed is a method of detecting a compound of interest, the method comprising bringing into contact a sample and a glmS riboswitch, wherein the riboswitch is activated by the compound of interest, wherein the riboswitch produces a signal when activated by the compound of interest, wherein the riboswitch produces a signal when the sample contains the compound of interest. The riboswitch can change conformation when activated by the compound of interest, wherein the change in conformation produces a signal via a conformation dependent label. The riboswitch can change conformation when activated by the compound of interest, wherein the change in conformation causes a change in expression of an RNA linked to the riboswitch, wherein the change in expression produces a signal. The signal can be produced by a reporter protein expressed from the RNA linked to the riboswitch.

Disclosed is a method comprising (a) testing a compound for inhibition of gene expression of a gene encoding an RNA comprising a riboswitch, wherein the inhibition is via the riboswitch, and (b) inhibiting gene expression by bringing into contact a cell and a compound that inhibited gene expression in step (a), wherein the cell comprises a gene encoding an RNA comprising a riboswitch, wherein the compound inhibits expression of the gene by binding to the riboswitch.

### A. Identification of Antimicrobial Compounds

Riboswitches are a new class of structured RNAs that have evolved for the purpose of binding small organic molecules. The natural binding pocket of riboswitches can be targeted with metabolite analogs or by compounds that mimic the shape-space of the natural metabolite. The small molecule ligands of riboswitches provide useful sites for derivitization to produce drug candidates. Distribution of some riboswitches is shown in Table 1 of U.S. Application Publication No. 2005-0053951. Once a class of riboswitch has been identified and its potential as a drug target assessed, such as the glmS riboswitch, candidate molecules can be identified.

The emergence of drug-resistant stains of bacteria highlights the need for the identification of new classes of antibiotics. Anti-riboswitch drugs represent a mode of anti-bacterial action that is of considerable interest for the following reasons. Riboswitches control the expression of genes that are critical for fundamental metabolic processes. Therefore manipulation of these gene control elements with drugs yields new antibiotics. These antimicrobial agents can be considered to be bacteriostatic, or bacteriocidal. Riboswitches also carry RNA structures that have evolved to selectively bind metabolites, and therefore these RNA receptors make good drug targets as do protein enzymes and receptors. Furthermore, it has been shown that two antimicrobial compounds (discussed above) kill bacteria by deactivating the antibiotics resistance to emerge through mutation of the RNA target.

As disclosed herein, the atomic-resolution structure model for a glmS riboswitch has been elucidated (Cochrane 2007, herein incorporated by reference in its entirety for its teaching concerning the glmS ribozyme structure), which enables the use of structure-based design methods for creating riboswitch-binding compounds. Specifically, the model for the binding site of the glmS riboswitch shows that two channels are present that would permit ligand modification (FIG. 2). GlcN6P analogs have been generated with chemical modifications at certain sites disclosed herein, and nearly all tested so far bind to the riboswitch with sub-nanomolar dissociation constants. FIG. 2 depicts the structures of GlcN6P analogs synthesized with modified chemical structures. Most of these compounds take advantage of the molecular recognition “blind spots” in the binding site model or the aptamer domain form a glmS riboswitch. The successful compounds can be used as a scaffold upon which further chemical variation can be introduced to create non-toxic, bioavailable, high affinity, anti-riboswitch compounds.

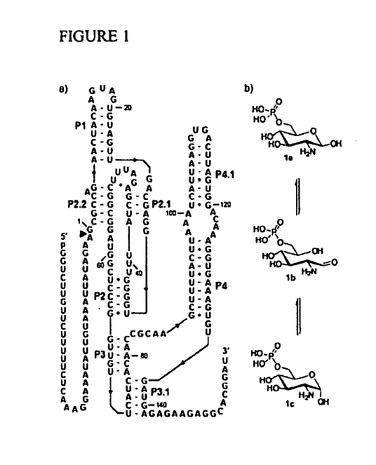

As seen in Example 1, the molecular recognition characteristics of the glmS ribozyme was found by determining the effects of GlcN6P and various GlcN6P analogs on the self-cleavage activity of a 200-nucleotide glmS ribozyme construct from B. cereus (FIG. 1). K_{D} values for each ligand were determined by plotting ribozyme rate constants versus ligand concentrations. Previous studies using similar methods revealed that the phosphate moiety of GlcN6P (FIG. 1b; 1a) is necessary for maximal affinity between ligand and glmS ribozyme (Winkler 2004; McCarthy 2005). The amine group of the ligand is also known to be essential for ribozyme function (Winkler 2004; McCarthy 2005). However, linear amine-containing compounds can induce modest ribozyme activity (McCarthy 2005), showing that acyclic (FIG. 1b) or alternative anomeric forms (FIG. 1c) of GlcN6P can be active. Therefore, a series of analogs were tested (FIG. 2) to probe the importance of structural conformation of GlcN6P and of individual functional groups on the pyranose ring.

Under physiological conditions, GlcN6P equilibrates between an acyclic form (FIG. 1b) and two cyclic β- (FIG. 1a) and α-anomer (FIG. 1c) forms (FIG. 1b) (Schray 1978). The relative amount of 1a and 1c in solution is 60:40 at 25° C. as determined by ¹H NMR in D₂O, with less than 1% in the acyclic form (Schray 1978). Each conformer could exhibit differences in RNA binding affinity and ribozyme activity similar to that observed for the GlmS protein (Teplyakov 1998).

Previous studies of the molecular recognition characteristics of other riboswitch classes have revealed that a high level of molecular discrimination can be achieved by natural ligand-binding RNAs (Lim 2006). Analogs that efficiently and selectively trigger glmS ribozyme cleavage can be used to disrupt the expression of GlmS metabolic enzymes in pathogenic bacteria, which can disrupt their normal cellular function.

In order to assess activity of given compounds, the following example can be used as a standard. Although the natural sequence of the glmS element forms a unimolecular cis-cleaving ribozyme, active glmS ribozyme can also be constructed as a biomolecular cis-acting ribozyme. In this format, the cleaved strand, termed the substrate, includes the 5′ base pairs that form half of the pairing element 1 (P1) and the conserved nucleotides upstream P1. The non-cleaved ribozyme strand includes the 3′ half of P1 and the remaining sequence of the glmS element. Using this biomolecular format, the 16-nucleotide substrate strand was labeled at the 3′ and the 5′ ends with the fluorescently probes fluorescein (F1) and cy3, respectively. In an uncleaved substrate RNA, emission of the excited state fluorescein is quenched by the enforced proximity of the cy3 quencher. Upon cleavage in the presence of the Gln6P system, the binding of Gln6P (or related derivatives) to the glmS riboswitch can be rapidly screened using standard high-throughput techniques.

After a minimum of 10 hours incubation, the fluorescence intensity of fluoroscein increased ˜4 fold in the presence of ˜160 μM glucosamine-6-phosphate, whereas no observable change was detected in the absence of Gln6P. For example, a compound can be identified as activating a riboswitch or can be determined to have riboswitch activating activity if the signal in a riboswitch assay is increased in the presence of the compound by at least 1 fold, 2 fold, 3 fold, 4 fold, 5 fold, 50%, 75%, 100%, 125%, 150%, 175%, 200%, 250%, 300%, 400%, or 500% compared to the same riboswitc assay in the absence of the compound (that is, compared to a control assay). The riboswitch assay can be performed using any suitable riboswitch construct. Riboswitch constructs that are particularly useful for riboswitch activation assays are described elsewhere herein. The identification of a compound as activating a riboswitch or as having a riboswitch activation activity can be made in terms of one or more particular riboswitches, riboswitch constructs or classes of riboswitches. For convenience, compounds identified as activating a glmS riboswitch or having riboswitch activating activity for a glmS riboswitch can be so identified for particular glmS riboswitches, such as the glmS riboswitches found in Bacillus anthracis, B. cereus, B. subtilis, Thermoanaerobacter tengcongensis, or S. aureus. When screening with high-throughput methods, the inclusion of 0.01% sodium dodecylsulfate was necessary for full ribozyme activity, consistent with previous demonstrations the utility of this detergent for preventing adhesion of small concentrations of the RNA to plastic tubes and plates. To further explore the discriminatory limits of this detection system, a library of sixteen Gln6P derivatives was also screened. The resulting glmS binding activities correspond well with the binding activities independently determined by gel electrophoresis methods (Table 1).

TABLE 1
Binding of Gln6P analogs to the glmS ribozyme measured by
high-throughput fluorescence screen or in-line probing

| | | Activity by |
|---|---|---|
| | | electrophoretic |
| Analog | Fl_{21 h}/Fl_{g h} | in-line probing |
| Gln6P | 3.36 | Yes |
| Ma6P | 2.89 | Yes |
| 1-dGln6P | 2.52 | Yes |
| Glnol6P | 0.80 | No |
| Gal6P | 1.69 | ND |
| β-MeO-Gln 6P | 1.02 | No |
| α-MeO-Gln 6P | 1.07 | No |
| MeN-Gln 6P | 3.49 | Yes |
| Me₃N-Gln 6P | 1.27 | No |
| Al6P | 3.27 | Yes |
| Gln6PS | 1.11 | No |
| 1-AmGln 6P | 1.00 | No |
| Me₃N-Gln | 1.17 | No |
| AcNGln 6P | 2.28 | Weakly |
| Gln6P com | 2.24 | Yes |
| Gln com | 1.87 | Yes |

### B. Methods of Using Antimicrobial Compounds

Disclosed herein are in vivo and in vitro anti-bacterial methods. By “anti-bacterial” is meant inhibiting or preventing bacterial growth, killing bacteria, or reducing the number of bacteria. Thus, disclosed is a method of inhibiting or preventing bacterial growth comprising contacting a bacterium with an effective amount of one or more compounds disclosed herein. Additional structures for the disclosed compounds are provided herein.

Disclosed herein is also a method of inhibiting growth of a cell, such as a bacterial cell, that is in a subject, the method comprising administering an effective amount of a compound as disclosed herein to the subject. This can result in the compound being brought into contact with the cell. The subject can have, for example, a bacterial infection, and the bacterial cells can be inhibited by the compound. The bacteria can be any bacteria, such as bacteria from the genus Bacillus or Staphylococcus, for example. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

“Inhibiting bacterial growth” is defined as reducing the ability of a single bacterium to divide into daughter cells, or reducing the ability of a population of bacteria to form daughter cells. The ability of the bacteria to reproduce can be reduced by about 10%, about 20%, about 30%, about 40%, about 50%, about 60%, about 70%, about 80%, about 90%, or 100% or more.

Also provided is a method of killing a bacterium or population of bacteria comprising contacting the bacterium with one or more of the compounds disclosed and described herein.

“Killing a bacterium” is defined as causing the death of a single bacterium, or reducing the number of a plurality of bacteria, such as those in a colony. When the bacteria are referred to in the plural form, the “killing of bacteria” is defined as cell death of a given population of bacteria at the rate of 10% of the population, 20% of the population, 30% of the population, 40% of the population, 50% of the population, 60% of the population, 70% of the population, 80% of the population, 90% of the population, or less than or equal to 100% of the population.

The compounds and compositions disclosed herein have anti-bacterial activity in vitro or in vivo, and can be used in conjunction with other compounds or compositions, which can be bacteriocidal as well.

By the term “therapeutically effective amount” of a compound as provided herein is meant a nontoxic but sufficient amount of the compound to provide the desired reduction in one or more symptoms. As will be pointed out below, the exact amount of the compound required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount may be determined by one of ordinary skill in the art using only routine experimentation.

The compositions and compounds disclosed herein can be administered in vivo in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions or compounds disclosed herein can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition or compounds, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The compositions and compounds disclosed herein can be used therapeutically in combination with a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Therapeutic compositions as disclosed herein may also be delivered by the use of monoclonal antibodies as individual carriers to which the compound molecules are coupled. The therapeutic compositions of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the therapeutic compositions of the present disclosure may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

Preferably at least about 3%, more preferably about 10%, more preferably about 20%, more preferably about 30%, more preferably about 50%, more preferably 75% and even more preferably about 100% of the bacterial infection is reduced due to the administration of the compound. A reduction in the infection is determined by such parameters as reduced white blood cell count, reduced fever, reduced inflammation, reduced number of bacteria, or reduction in other indicators of bacterial infection. To increase the percentage of bacterial infection reduction, the dosage can increase to the most effective level that remains non-toxic to the subject.

As used throughout, “subject” refers to an individual. Preferably, the subject is a mammal such as a non-human mammal or a primate, and, more preferably, a human. “Subjects” can include domesticated animals (such as cats, dogs, etc.), livestock (e.g., cattle, horses, pigs, sheep, goats, etc.), laboratory animals (e.g., mouse, rabbit, rat, guinea pig, etc.) and fish.

A “bacterial infection” is defined as the presence of bacteria in a subject or sample. Such bacteria can be an outgrowth of naturally occurring bacteria in or on the subject or sample, or can be due to the invasion of a foreign organism.

The compounds disclosed herein can be used in the same manner as antibiotics. Uses of antibiotics are well established in the art. One example of their use includes treatment of animals. When needed, the disclosed compounds can be administered to the animal via injection or through feed or water, usually with the professional guidance of a veterinarian or nutritionist. They are delivered to animals either individually or in groups, depending on the circumstances such as disease severity and animal species. Treatment and care of the entire herd or flock may be necessary if all animals are of similar immune status and all are exposed to the same disease-causing microorganism.

Another example of a use for the compounds includes reducing a microbial infection of an aquatic animal, comprising the steps of selecting an aquatic animal having a microbial infection, providing an antimicrobial solution comprising a compound as disclosed, chelating agents such as EDTA, TRIENE, adding a pH buffering agent to the solution and adjusting the pH thereof to a value of between about 7.0 and about 9.0, immersing the aquatic animal in the solution and leaving the aquatic animal therein for a period that is effective to reduce the microbial burden of the animal, removing the aquatic animal from the solution and returning the animal to water not containing the solution. The immersion of the aquatic animal in the solution containing the EDTA, a compound as disclosed, and TRIENE and pH buffering agent may be repeated until the microbial burden of the animal is eliminated. (U.S. Pat. No. 6,518,252).

Other uses of the compounds disclosed herein include, but are not limited to, dental treatments and purification of water (this can include municipal water, sewage treatment systems, potable and non-potable water supplies, and hatcheries, for example).

### Specific Embodiments

Disclosed herein is a method of inhibiting gene expression, the method comprising (a) bringing into contact a compound and a cell, (b) wherein the compound has the structure of Formula I:

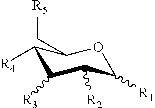

or pharmaceutically acceptable salts thereof, physiologically hydrolyzable and acceptable esters thereof, or both, wherein R₁ is H, OH, SH, NH₂, or CH₃, wherein R₂ is NH—R₆, wherein R₆ is H, CH₃, C₂H₅, n-propyl, C(O)CH₃, C(O)C₂H₅, C(O)n-propyl, C(O)iso-propyl, C(O)OCH₃, C(O)OC₂H₅, C(O)NH₂, or NH₂, wherein R₃ is H, OH, SH, NH₂, or CH₃, wherein R₄ is a hydrogen bond donor, wherein R₅ is a hydrogen bond acceptor, and wherein the compound is not glucosamine-6-phosphate, wherein the cell comprises a gene encoding an RNA comprising a glmS riboswitch, wherein the compound inhibits expression of the gene by binding to the glmS riboswitch.

Also disclosed herein is a method of inhibiting gene expression, the method comprising bringing into contact a compound as disclosed above and a cell, wherein the cell comprises a gene encoding an RNA comprising a glmS-responsive riboswitch, wherein the compound inhibits expression of the gene by binding to the glmS-responsive riboswitch. The cell can be a bacterial cell, for example, and the compound can kill or inhibit the bacterial cell. The cell can contain a glmS riboswitch. The cell can be Bacillus or Staphylococcus.

Also disclosed is a method of inhibiting gene expression in a cell and/or inhibiting cell growth in a subject containing the cell by bringing into contact a compound and the cell by administering the compound to a subject. In some forms of the method, the compound is not a substrate for enzymes of the subject that have glucosamine-6-phosphate as a substrate. A compound is not a substrate of an enzyme if less than 1%, 2%, 3%, 4%, or 5% of the compound is altered or metabolozed by the enzyme for a length of time and under conditions in which the enzyme alters or metabolizes 80% or more of its primary substrate. A primary substrate for an enzyme is the normal biological substrate for the enzyme upon which the enzyme has the highest enzymatic activity. In some forms of the method, the compound is not a substrate for enzymes of the subject that alter glucosamine-6-phosphate. In some forms of the method, the compound is not a substrate for enzymes of the subject that metabolize glucosamine-6-phosphate. In some forms of the method, the compound is not a substrate for enzymes of the subject that catabolize glucosamine-6-phosphate. In some forms of the method, the cell is a bacterial cell in the subject, wherein the compound kills or inhibits the growth of the bacterial cell. In some forms of the method, the subject has a bacterial infection. In some forms of the method, the compound is administered in combination with another antimicrobial compound. In some forms of the method, the compound inhibits bacterial growth in a biofilm.

Disclosed herein is a compound having the structure of Formula I:

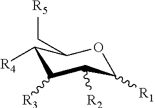

or pharmaceutically acceptable salts thereof, physiologically hydrolyzable and acceptable esters thereof, or both, wherein R₁ is H, OH, SH, NH₂, or CH₃, wherein R₂ is NH—R₆, wherein R₆ is H, CH₃, C₂H₅, n-propyl, C(O)CH₃, C(O)C₂H₅, C(O)n-propyl, C(O)iso-propyl, C(O)OCH₃, C(O)OC₂H₅, C(O)NH₂, or NH₂, wherein R₃ is H, OH, SH, NH₂, or CH₃, wherein R₄ is a hydrogen bond donor, wherein R₅ is a hydrogen bond acceptor, and wherein the compound is not glucosamine-6-phosphate. Also disclosed are compounds in which R₄ is OH, SH, NH₂, NH₃+, CH₂OH, CH(OH)CH₃, CH₂CH₂OH, CH₂SH, CH(SH)CH₃, CH₂CH₂SH, CH₂NH₂, CH(NH₂)CH₃, CH₂CH₂NH₃, CO₂H, CONH₂, CONHalkyl, ═NH, ═NOH, ═NSH, ═NCO₂H, ═CH₂, CH═NH, CH═NOH, CH═NSH, CH═NCO₂H, OCH₂OH, OCH₂CH₂OH, PhOH, NHalkyl, NHNH₂, NHNHalkyl, NHCOalkyl, NHCO₂alkyl, NHCONH₂, NHSO₂alkyl, or NHOalkyl. Also disclosed are compounds in which R₄ is not OH when R₁ is H or OH and R₂ is NH₂ or NHCH₃. Further disclosed are compounds in which R₅ is OP(O)(OH)₂, OP(S)(OH)₂, OP(O)OHSH, OS(O)₂OH, or OS(O)₂SH. Also disclosed are compounds in which R₅ is OS(O)₂OH or OS(O)₂SH. Furthermore, R₅ can be negatively charged. Further disclosed are compounds in which R₅ is ═O, CO₂R₉, OCO₂R₉, OCH₂OR₉, OC₂H₅OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, PO₂(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, and wherein R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), or —C(CH₃)₃, —CF₃. Also disclosed are compounds in which R₅ is ═O, OH, OR₉, COR₉, CN, NO₂, tetrazole, SOR₉, N(R₉)₂, CO₂R₉, OCO₂R₉, OCH₂OR₉, OC₂H_{S}OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, PO₂(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, and R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), —C(CH₃)₃, or —CF₃. Also disclosed are compounds in which R₄ is NH₂, NH₃⁺, OH, SH, NOH, NHNH₂, NHNH₃⁺, CO₂H, SO₂OH, B(OH)₂, or imidazolium. Also disclosed are compounds in which R₄ is NH₂, NH₃⁺, SH, NOH, NHNH₂, NHNH₃⁺, CO₂H, SO₂OH, B(OH)₂, or imidazolium. In one example, the cell has been identified as being in need of inhibited gene expression. The cell can be a bacterial cell, and the compound can kill or inhibit the growth of the bacterial cell. The compound can be bound to a glmS riboswitch. The compound can bind to a glmS riboswitch. The compound can activate a glmS riboswitch.

Further disclosed is a composition comprising the compound described above and a regulatable gene expression construct comprising a nucleic acid molecule encoding an RNA comprising a glmS riboswitch operably linked to a coding region, wherein the glmS riboswitch regulates expression of the RNA, wherein the glmS riboswitch and coding region are heterologous. The glmS riboswitch can produce a signal when activated by the compound. For example, the riboswitch can change conformation when activated by the compound, and the change in conformation can produce a signal via a conformation dependent label. Furthermore, the riboswitch can change conformation when activated by the compound, wherein the change in conformation causes a change in expression of the coding region linked to the riboswitch, wherein the change in expression produces a signal. The signal can be produced by a reporter protein expressed from the coding region linked to the riboswitch.

Also disclosed is a method comprising: (a) testing the compound as described above for inhibition of gene expression of a gene encoding an RNA comprising a glmS riboswitch, wherein the inhibition is via the glmS riboswitch, and (b) inhibiting gene expression by bringing into contact a cell and a compound that inhibited gene expression in step (a), wherein the cell comprises a gene encoding an RNA comprising the glmS riboswitch, wherein the compound inhibits expression of the gene by binding to the glmS riboswitch.

Also disclosed is the atomic structure of a natural glmS-responsive riboswitch comprising an atomic structure comprising the atomic coordinates listed in Table 2. Also disclosed are the atomic structure of the active site and binding pocket as depicted in FIG. 9 and the atomic coordinates of the active site and binding pocket depicted in FIG. 9 contained within Table 2.

Further disclosed is a method of identifying a compound that interacts with a riboswitch comprising: modeling the atomic structure of a glmS riboswitch with a test compound; and determining if the test compound interacts with the riboswitch. Furthermore, determining if the test compound interacts with the riboswitch can comprise determining a predicted minimum interaction energy, a predicted bind constant, a predicted dissociation constant, or a combination, for the test compound in the model of the riboswitch. Determining if the test compound interacts with the riboswitch can comprise determining one or more predicted bonds, one or more predicted interactions, or a combination, of the test compound with the model of the riboswitch. Atomic contacts can be determined, thereby determining the interaction of the test compound with the riboswitch. The method of identifying a compound that interacts with a riboswitch can further comprise the steps of: identifying analogs of the test compound; and determining if the analogs of the test compound interact with the riboswitch.

Further disclosed is a method of killing bacteria, comprising contacting the bacteria with an analog identified by the method disclosed above. Further disclosed is a method of killing bacteria, comprising contacting the bacteria with a compound identified by the method disclosed above. A gel-based assay or a chip-based assay can be used to determine if the test compound interacts with the riboswitch. The test compound can interact via van der Waals interactions, hydrogen bonds, electrostatic interactions, hydrophobic interactions, or a combination. The riboswitch can comprise an RNA cleaving ribozyme, for example. A fluorescent signal can be generated when a nucleic acid comprising a quenching moiety is cleaved. Molecular beacon technology can be employed to generate the fluorescent signal. The methods disclosed herein can be carried out using a high throughput screen.

Disclosed herein is also a method of inhibiting growth of a cell, such as a bacterial cell, that is in a subject, the method comprising administering an effective amount of a compound as disclosed herein to the subject. This can result in the compound being brought into contact with the cell. The subject can have, for example, a bacterial infection, and the bacterial cells can be the cells to be inhibited by the compound. The bacteria can be any bacteria, such as bacteria from the genus Bacillus or Staphylococcus, for example. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

### Examples

### A. Example 1

### Characteristics of Ligand Recognition by a glmS Self-cleaving Ribozyme

The glmS ribozyme (Winkler 2004; Barrick 2004; McCarthy 2005; Wilkinson 2005; Soukup 2006; Roth 2006; Jansen 2006) from Bacillus cereus is a representative of a unique riboswitch (Mandal 2004; Winkler 2005) class whose members undergo self-cleavage with accelerated rate constants when bound to glucosamine-6-phosphate (GlcN6P). These metabolite-sensing ribozymes are found in numerous Gram-positive bacteria where they control expression of the glmS gene. The glmS gene product (glutamine-fructose-6-phosphate amidotransferase) generates GlcN6P (Badet-Denisot 1993; Milewski 2002) which binds to the ribozyme and triggers self-cleavage by internal phosphoester transfer (Winkler 2004). The ribozyme is embedded within the 5′ untranslated region (UTR) of the glmS messenger RNA and self-cleavage prevents GlmS protein production, thereby decreasing the concentration of GlcN6P. The combination of molecular sensing, self-cleavage, and gene control functions allows this small RNA to operate both as a ribozyme and as a riboswitch.

It has been shown that the glmS ribozyme from B. cereus, and the homologous ribozyme from Bacillus subtilis respond to GlcN6P with an apparent dissociation constant (K_{D}) of ˜200 μM (Winkler 2004; McCarthy 2005; Roth 2006). Although this K_{D} value is greater than those determined for most other natural riboswitches, glmS ribozymes exhibit a high level of molecular recognition specificity. For example, glucosamine-6-sulphate can induce ribozyme activation to the same extent as GlcN6P, albeit when present at concentrations that are ˜100-fold greater. In contrast, glucose-6-phosphate, wherein the 2-amine group of GlcN6P is replaced with a hydroxyl group, completely fails to trigger ribozyme action (Winkler 2004; McCarthy 2005).

Riboswitches must be capable of discriminating against compounds related to their natural ligands to prevent undesirable regulation of metabolic genes. However, it is possible to generate analogs that trigger riboswitch function and inhibit bacterial growth, as has been demonstrated for riboswitches that normally respond to lysine (Sudarsan 2003) and thiamine pyrophosphate (Sudarsan 2006). Proper expression of the GlmS protein is critical for bacterial viability (Badet-Denisot 1993; Milewski 2002), and analogs of GlcN6P that interfere with normal gene expression by triggering glmS ribozyme activity can serve as antimicrobial agents. Therefore, an increased understanding of the molecular recognition characteristics of glmS ribozymes was sought.

The molecular recognition characteristics of the glmS ribozyme was found by determining the effects of GlcN6P and various GlcN6P analogs on the self-cleavage activity of a 200-nucleotide glmS ribozyme construct from B. cereus (FIG. 1). K_{D} values for each ligand were determined by plotting ribozyme rate constants versus ligand concentrations (Experimental Section). Previous studies using similar methods revealed that the phosphate moiety of GlcN6P (FIG. 1b; 1a) is necessary for maximal affinity between ligand and glmS ribozyme (Winkler 2004; McCarthy 2005). The amine group of the ligand is also known to be essential for ribozyme function (Winkler 2004; McCarthy 2005). However, linear amine-containing compounds can induce modest ribozyme activity (McCarthy 2005), suggesting that acyclic (1b) or alternative anomeric forms (1c) of GlcN6P might be active. Therefore, a series of analogs were tested (FIG. 2) to probe the importance of structural conformation of GlcN6P and of individual functional groups on the pyranose ring.

Under physiological conditions, GlcN6P equilibrates between an acyclic form (1b) and two cyclic β- (1a) and α-anomer (1c) forms (FIG. 1b) (Schray 1978). The relative amount of 1a and 1c in solution is 60:40 at 25° C. as determined by ¹H NMR in D₂O, with less than 1% in the acyclic form (Schray 1978). Each conformer could exhibit differences in RNA binding affinity and ribozyme activity similar to that observed for the GlmS protein (Teplyakov 1998).

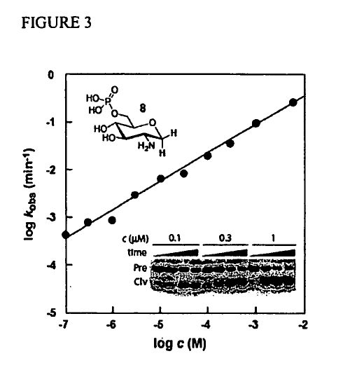

Small molecules such as serinol and ethanolamine promote ribozyme activity, although they are orders of magnitude less effective than GlcN6P (McCarthy 2005). Similarly, the acyclic analog 3 has no detectable activity under the assay conditions used in the current study (FIG. 2b, Experimental Section). In contrast, the cyclic analog 8 lacking the hydroxyl group at position 1 activates ribozyme self-cleavage to ˜ 1/70^{th} of the activity exhibited by GlcN6P (FIG. 2c, FIG. 3). These results demonstrate that alteration of the chemical structure at position 1 of the pyranose ring has only a modest effect on ribozyme activity, but opening of the ring at this position (as in 3 and most likely in 1b) is far more deleterious.

Because 1b is unlikely to be relevant for normal function of the ribozyme, one or both of the anomers of GlcN6P must serve as the activator. Analogs of 1a (5) and 1c (6) were tested, wherein the stereochemistry of the analogs are maintained by methylation of the oxygen atoms at the 1 position. The current results show the ribozyme forms a tight binding pocket near the 1 position that precludes analog binding. Since 8 can activate ribozyme cleavage substantially, the modification at position 1 might only modestly disrupt a molecular interaction between ligand and ribozyme. Alternatively, the influence of the 1-hydroxyl group on the pKₐ of the 2-amine group can also be the cause of the reduction in activity of 8. For example, ethylamine (pKₐ=10.7) has a higher pKₐ than ethanolamine (pKₐ=9.50), (Lide 1994) indicating that an adjacent hydroxyl group can reduce the basicity of an amine by more than one unit. Therefore the change in pKₐ caused by the absence of the 1-hydroxyl group in 8 could disrupt either ligand binding or ribozyme catalysis.

Compounds 2 and 13 were examined to determine the importance of the 3- and 4-hydroxyl groups for ribozyme activation. While 13 exhibits activity that is similar to GlcN6P, 2 does not induce activity under the assay conditions used (FIGS. 2b and 2c). These results imply that the 4-hydroxyl group is critical for binding. In contrast, 3-hydroxyl group might have only a modest impact on binding or otherwise might influence reactivity via an inductive effect on the 2-amine group.

Replacement of the phosphate group with sulfate reduces affinity for the ligand by ˜100 fold (Winkler 2004). However, removal of the phosphate group (glucosamine) causes an even greater loss of ligand affinity. To further assess the importance of phosphate oxygen atoms, the phosphorothiolate analog 4 was generated. This change also reduces the rate constant of ribozyme cleavage by approximately two orders of magnitude compared to 1. However, GlcN6P is bound by the ribozyme >1000-fold more tightly than is glucosamine (Winkler 2004; McCarthy 2005; Lide 1994; Mayer 2006). Although the phosphate modifications tested can only partially disrupt a single interaction between ligand and ribozyme, it is likely that more than one binding interaction is made to this part of the ligand.

The 2-amine group, or an analogous amine is present in all compounds that induce ribozyme activity (Winkler 2004; McCarthy 2005). Therefore a series of structural and stereochemical isomers of 1 were tested wherein this functional group was altered. The interchange of 1-hydroxyl and 2-amine groups in 7 does not support ribozyme cleavage, suggesting the location of the 2-amine group is critical for activity. The ribozyme is activated by 9 with only a modest reduction in efficiency compared to GlcN6P, despite the steric hindrance that might be caused by the methyl group. In contrast, 10 and 11 are inactive, showing that the ability of the amine to accept or donate protons for bonding or catalysis is essential.

Compound 12 carries an amine group at the 2 position with opposing stereochemical configuration and surprisingly induces cleavage to 1/35^{th} of the natural ligand. 12 can bind using the same contacts that are used to bind GlcN6P, but the relocation of the amine group in this pocket only slightly detracts from its ability to bind or to participate in proton-transfer-mediated catalysis.

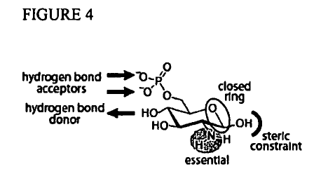

Based on these findings, a series of molecular recognition determinants for GlcN6P binding have been determined (FIG. 4). The 6-phosphate and 4-hydroxyl groups can serve as hydrogen-bond donor and acceptor sites. Although the non-bridging oxygen atoms of a phosphate group can interact with metal ions via inner-sphere coordination, this is unlikely for this RNA because the ribozyme can attain full activity when Mg²⁺ ions are replaced with cobalt hexamine (Roth 2006). Cobalt hexamine simulates fully-hydrated Mg²⁺ ions, and can only form hydrogen bonds with an adjacent phosphate.

The reduced activity for 8 can be due to the expected increase in the pKₐ of the amine, which can influence its ability to function in proton transfer reactions. The loss of activity observed with 8 is can be due to a shift in amine pKₐ or due at least in part to disruption of a molecular recognition contact. GlcN6P can function as a cofactor for RNA cleavage (Winkler 2004) and nucleic acid enzymes that use small molecules presumably to assist in proton transfer have been identified previously. Both the absence of ligand-induced shape change in the RNA (Roth 1998) and pH profile changes brought about by the use of various ligand analogs (McCarthy 2005) show that GlcN6P directly participates in the chemical step of the reaction.

If the amine group of GlcN6P is a key moiety in the ribozyme active site, then the simplest explanation for the data is that the ligand serves as a general base catalyst. The logarithm of k_{obs} for ribozyme activity with increasing pH increases linear with a slope of 1. Furthermore, GlcN6P analogs that exhibit higher pKₐ values for the amine group are less effective inducers of ribozyme activity (8) or exhibit an increase in the pH required to reach half-maximal ribozyme activity (McCarthy 2005). Although other more complex mechanisms are possible, the ribozyme can use GlcN6P to assist in deprotonation of the 2′-hydroxyl group at the labile internucleotide linkage (Roth 2006).

Previous studies of the molecular recognition characteristics of other riboswitch classes have revealed that a high level of molecular discrimination can be achieved by natural ligand-binding RNAs (Lim 2006). Analogs that efficiently and selectively trigger glmS ribozyme cleavage can be used to disrupt the expression of GlmS metabolic enzymes in pathogenic bacteria, which can disrupt their normal cellular function.

### Experimental Section

The glmS ribozyme from B. cereus (FIG. 1a) was generated by in vitro transcription as described previously, (Roth 2006) 5′-³²P-radiolabeled, (Lim 2006) and purified by PAGE. Rate constants were established using methods and reaction conditions similar to those described previously (Roth 2006) with the exception that reaction mixtures contained 50 mM HEPES buffer (pH 7.5 at 23° C.) in place of Tris-HCl buffer. Ligand concentrations and incubation times used are defined for each assay. Ribozyme activity was established by quantitating the amounts of cleaved and uncleaved RNAs using a Typhoon imager (Amersham Biosciences).

General Methods: Reagents obtained from commercial suppliers were used without further purification unless otherwise noted. Melting points were determined with an Electrothermal capillary melting point apparatus and the values reported are uncorrected. NMR (¹H, ¹³C and ³¹P) spectra were recorded (Bruker AMX-400 MHz or Bruker Advance DRX-500 MHz) as noted, referenced to D₂O (4.79 ppm for ¹H) or CDCl₃ (7.27 ppm for ¹H and 77.0 ppm for ¹³C). Analytical thin-layer chromatography was performed using E. Merck silica gel 60 F₂₅₄ plates. UV-inactive compounds were visualizing by dipping the plates in ninhydrin solution and heating. Synthetic DNAs were purchased from the HHMI Keck Foundation Biotechnology Resource Center at Yale University (New Haven, Conn., USA).

Materials: The following compounds or reagents were commercially available from the indicated sources: D-(+)-glucosamine hydrochloride, D-glucose 6-phosphate, hexokinase from Sacchromyces cerevisiae (Sigma); N-acetyl-D-glucosamine 6-phosphate, D-mannosamine hydrochloride, D-galactosamine hydrochloride, 2-acetamido-2-deoxy-β-D-glucopyranose 1,3,4,6-tetraacetate, 2-acetamido-2-deoxy-α-D-glucopyranosyl chloride, phosphoryl chloride, thiophosphoryl chloride, adenosine 5′-triphosphate (Aldrich); 1,3,4,6-tetra-O-acetyl-2-amino-2-desoxy-β-D-glucopyranose hydrochloride (Oakwood); 2-deoxy-2-(trimethylammonio)-D-glucose (Timtec). Compounds synthesized for this study as described below include 2-amino-2-deoxy-D-glucitol-6-phosphate (Beame 1996) 2-amino-2-deoxy-D-mannose 6-phosphate (Liu 2001) 2-deoxy-2-amino-D-allose (Jeanioz 1957), 2-deoxy-2-methylamino-D-glucose, (Gorin 1971), 2-amino-1,5-anhydro-2-deoxyglucitol hydrochloride (Schaefer 1998), methyl 2-amino-2-deoxy-α-D-glucose 6-phosphate (Ohno 1981), methyl 2-amino-2-deoxy-β-D-glucose 6-phosphate (Ohno 1981) and 2-amino-2-deoxy-D-galactose 6-phosphate (Distler 1958).

2-amino-2-deoxy-1,3,4,6-tetra-O-(trimethylsilyl)-α-D-glucopyranose. 2-amino-2-deoxy-1,3,4,6-tetra-O-(trimethylsilyl)-α-D-glucopyranose was prepared by a modification of the procedure of Gautheron (Auge 1998), D-glucosamine hydrochloride (1.0 g, 4.64 mmol) was dissolved in 45 mL of pyridine and treated with 7.0 mL (33.39 mmol) of hexamethyldisilazane and 3.5 mL (27.82 mmol) of chlorotrimethylsilane. The mixture was heated at 60° C. for 3 h and filtered. The filtrate was partitioned between n-hexane and water, and the organic phase was separated. The aqueous phase was extracted with n-hexane, and the combined organic phase was washed with 1 N HCl, dried over MgSO₄, and concentrated in vacuo to give the desired product as yellowish white solid. 100% yield; ¹H NMR (CDCl₃, 400 MHz) δ5.10 (d, 1H, J=3.2 Hz, 1-H), 3.68 (m, 3H, 3-, 4-, and 5-H), 3.49 (m, 2H, 6-H), 2.51 (dd, 1H, 2-H), 0.0-0.2 (s, 36H, 4 (CH₃)₃Si); ¹³C NMR (CDCl₃, 100 MHz) δ95.0, 77.9, 73.3, 72.4, 62.4, 57.8, 1.7, 1.2, 0.3, 0.1; IR (neat, cm⁻¹); MS (ESI) m/e 468.0 ([M+H]⁺, C₁₈H₄₅NO₅Si₄ requires 467.9).

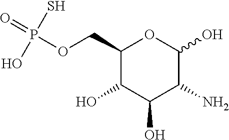

2-amino-2-deoxy-D-glucose 6-thiophosphate. To a mixture of 2-amino-2-deoxy-1,3,4,6-tetra-O-(trimethylsilyl)-α-D-glucopyranose (357 mg, 0.76 mmol) in 2 mL of toluene and 140 μL (1.68 mmol) of pyridine was added thiophosphoryl chloride (158 μL, 1.53 mmol), and the reaction mixture was heated at 30° C. for 16 h. The mixture was concentrated, dissolved in ethanol, and then coevaporated in vacuo. The crude mixture was treated with water and heated at 60° C. for 16 h, prior to being concentrated. The residue was coevaporated with water three times. Crystallization with ethanol and diethylether give the desired product as a white solid. mp 185-187° C. dec; ¹HNMR (D₂O, 500 MHz) δ, ¹³C NMR (D₂O, 125 MHz) ε; ³¹P NMR (D₂O, 162 MHz) δ41.9; IR (neat, cm⁻¹) 3363, 1028, 722; MS (ESI) m/e 275.1 ([M+H]⁺, C₆H₁₄NO₇PS requires 275.2).

General procedure for enzymatic phosphorylations by Saccharomyces cerevisiae hexokinase. The procedure was essentially based on that of Liu and Lee (2001) hexosamine hydrochloride (2.00 mmol), magnesium chloride hexahydrate (1.40 mmol), and adenosine 5′-triphosphate (2.20 mmol) were dissolved in 54 mL of distilled water. The solution was adjusted to pH 7.5 with 0.5 N KOH, and hexokinase (1320 U) dissolved in 1.0 mL water was added. The reaction mixture was continuously stirred for 2-3 h at ambient temperature. During the reaction, the pH of the solution was maintained at 7.5 by addition of 0.5 N KOH each half-hour until the pH became stable. TLC (2:1:1 n-BuOH—HOAc—H₂O) showed that the starting material completely disappeared, and a new compound appeared as a ninhydrin-positive and UV-negative spot. The mixture was then adjusted to pH 2.0 by addition of concentrated hydrochloric acid and a one third portion was loaded onto a column of Dowex 50 W×8 (H⁺ from, 200-400 mesh) cation exchange resin (2.5×25 cm). The column was eluted with water, and the major anions were immediately eluted followed by hexosamine 6-phosphate. The fractions containing the desired product were pooled and concentrated with a rotary evaporator below 45° C. and then lyophilized to give the product.

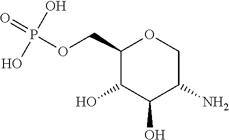

2-amino-1,5-anhydro-2-deoxyglucitol 6-phosphate. 2-deoxy-1,5-anhydro-2-deoxyglucitol 6-phosphate was prepared by the procedure of Liu and Lee (2001) mp 185-187° C. dec; ¹H NMR (D₂O, 500 MHz) δ4.07 (dd, 1H, H-1), 3.72 (dd, 1H, H-6), 3.64 (dd, 1H, H-6), 3.50 (dd, 1H, H-3), 3.41 (dd, 1H, H-1), 3.29 (m, 2H, H4 & H-5), 3.15 (m, 1H, H-2); ¹³C NMR (D₂O, 125 MHz) δ81.0, 74.6, 70.2, 66.4, 61.1, 51.9; ³¹P NMR (D₂O, 162 MHz) δ4.10; MS (ESI) m/e 243.4 ([M+H]⁺, C₆H₁₄NO₇P requires 243.2).

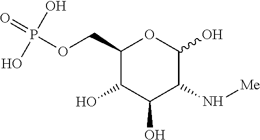

2-methylamino-2-deoxyglucose 6-phosphate. 2-methylamino-2-deoxyglucose 6-phosphate was prepared by the procedure of Liu and Lee (2001) mp 185-187° C. dec; ¹H NMR (D₂O, 500 MHz) δ5.21 (d, 1H, H-1), 4.05 (d, 3H, CH₃), 3.67 (m, 3H, 3-H, 4-H & 5-H) 3.34 (m, 2H, 6-H), 3.09 (m, 1H, H-2); ¹³C NMR (D₂O, 125 MHz) δ93.2, 89.6, 72.2, 71.1, 69.5, 64.1, 54.6; ³¹P NMR (D₂O, 162 MHz) δ4.59; MS (ESI) m/e 273.4 ([M+H]⁺, C₇H₁₆NO₈P requires 273.2).

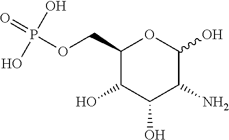

2-amino-2-deoxyallose 6-phosphate. 2-amino-2-deoxyallose 6-phosphate was prepared by the procedure of Liu and Lee.^{[2]} mp 185-187° C. dec; ¹H NMR (D₂O, 500 MHz) δ5.27 (d, 1H, H-1β), 4.85 (d, 1H, H-1α), 4.04 (m, 3H, H-3, H-4 & H-5), 3.50 (m, 2H, H-6), 3.23 (dd, 1H, H-2); ¹³C NMR (D₂O, 125 MHz) δ91.2, 88.6, 70.5, 68.5, 63.4, 58.1; ³¹P NMR (D₂O, 162 MHz) δ 4.38; MS (ESI) m/e 259.1 ([M+H]⁺, C₆H₁₄NO₈P requires 259.2).

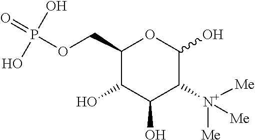

2-(trimethylammonio)-2-deoxyglucose 6-phosphate. 2-(trimethylammonio)-2-deoxy-glucose 6-phosphate was prepared by the procedure of Distler (1958) mp 185-187° C. dec; ¹H NMR (D₂O, 500 MHz) δ5.24 (s, 1H, H-1), 3.55 (m, 3H, H-3, H-4 & H-5), 3.50 (s, 9H, N(CH₃)₃), 3.14 (s, 2H, H-6); ¹³C NMR (D₂O, 125 MHz) δ95.6, 91.8, 75.5, 74.0, 71.2, 69.4; ³¹P NMR (D₂O, 162 MHz) δ3.40; MS (ES1) m/e 303.1 ([M+H]⁺, C₉H₂₁NO₈P require 302.2).

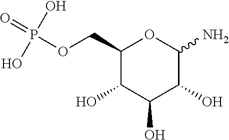

1-amino-1-deoxy 6-phosphate. 1-amino-1-deoxyglucose 6-phosphate was prepared by the modification of the procedure of Gallop (Vetter 1995). To a solution of D-glucose 6-phopsphate in saturated aqueous ammonium carbonate was stirred at room temperature for 5 days. Solid ammonium carbonate was added in fractions during the course of the reaction to ensure saturation. The mixture was loaded onto a column of Dowex 50 W×8 (H⁺ from, 200-400 mesh) cation exchange resin (2.5×25 cm). The column was eluted with water, and the major anions were immediately eluted, followed by hexosamine 6-phosphate. The fractions containing the desired product were pooled and concentrated with a rotary evaporator below 45° C. and then lyophilized to give the product. mp 185-187° C. dec; ¹H NMR (D₂O, 500 MHz) δ5.27 (d, 1H, H-1β), 4.85 (d, 1H, H-1α), 4.04 (m, 3H, H-3, H-4 & H-5), 3.50 (m, 2H, H-6), 3.23 (dd, 1H, H-2); ¹³C NMR (D₂O, 125 MHz) δ95.6, 91.8, 75.5, 74.0, 71.2, 69.4; ³¹P NMR (D₂O, 162 MHz) δ 3.40; MS (ESI) m/e 259.1 ([M+H]⁺, C₆H₁₄NO₈P requires 259.2).

Lengthy table referenced here
US20100324123A1-20101223-T00001
Please refer to the end of the specification for access instructions.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a riboswitch” includes a plurality of such riboswitches, reference to “the riboswitch” is a reference to one or more riboswitches and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### REFERENCES

[1] W. C. Winkler, A. Nahvi, A. Roth, J. A. Collins, R. R. Breaker, Nature 2004, 428, 281-286.
[2] J. E. Barrick, et al., Proc. Natl. Acad. Sci. USA 2004, 101, 6421-6426.
[3] T. J. McCarthy, M. A. Plog, S. A. Floy, J. A. Jansen, J. K. Soukup, G. A. Soukup, Chem. Biol. 2005, 12, 1221-1226.
[4] S. R. Wilkinson, M. D. Been, RNA 2005, 11, 1788-1794.
[5] G. A. Soukup, Nucleic Acids Res. 2006, 34, 968-975.
[6] A. Roth, A. Nahvi, M. Lee, I. Jona, R. R. Breaker, RNA 2006, 12, 607-619.
[7] J. A. Jansen, T. J. McCarthy, G. A. Soukup, J. K. Soukup. Nat. Struct. Mol. Biol. 2006 13, 517-523.
[8] M. Mandal, R. R. Breaker, Nat. Rev. Mol. Cell Bio. 2004, 5, 451-463.
[9] W. C. Winkler, R. R. Breaker, Annu. Rev. Microbio!. 2005, 59, 487-517.
[10] M.-A. Badet-Denisot, L. René, B. Badet, Bull. Soc. Chim. Fr. 1993, 130, 249-255.
[11] S. Milewski, Biochim. Biophys. Acta 2002, 1597, 173-192.
[12] N. Sudarsan, J. K. Wickiser, S. Nakamura, M. S. Ebert, R. R. Breaker, Genes Dev. 2003, 17, 2688-2697.
[13] N. Sudarsan, S. Cohen-Chalamish, S. Nakamura, G. M. Emilsson, R. R. Breaker, Chem. Biol. 2006, 12, 1325-1335.
[14] The synthetic details and spectral data are available in Supporting Information.
[15] K. J. Schray, S. J. Benkovic, Acc. Chem. Res. 1978, 11, 136-141.
[16] The atomic structure of GlmS bound to GlcN6P reveals a preference for the □-pyranose form; A. Teplyakov, G. Obmolova. M.-A. Badet-Denisot, B. Badet, I. Polikarpov, Structure 1998, 6, 1047-1055.
[17] D. R. Lide, CRC Handbook of Chemistry and Physics, 75th ed., CRC Press, Boca Raton, Fla. 1994, pp. 8-45.
[18] G. Mayer, M. Famulok, Chembiochem 2006, 7, 602-604.
[19] K. F. Blount, I. Puskarz, R. Penchovsky, R. R. Breaker, RNA Biology 2006, (in press).
[20] A. Roth, R. R. Breaker, Proc. Natl. Acad. Sci. USA 1998, 95, 6027-6031.
[21] K. J. Hampel, M. M. Tinsley, Biochemistry 2006, 45, 7861-7871.
[22] J. Lim, W. C. Winkler, S. Nakamura, V. Scott, R. R. Breaker, Angewandte Chem. Int. Ed. 2006, 45, 964-968; Angew Chem. 2006, 118, 978-982.
[23] J. C. Cochrane, S. V. Lipchock, S. A. Strobel, Chem Biol 2007, 14, 97-105.

LENGTHY TABLES
The patent application contains a lengthy table section. A copy of the table is available in electronic form from the USPTO web site (http://seqdata.uspto.gov/?pageRequest=docDetail&DocID=US20100324123A1). An electronic copy of the table will also be available from the USPTO upon request and payment of the fee set forth in 37 CFR 1.19(b)(3).

We claim:

1. A method of inhibiting gene expression, the method comprising
(a) bringing into contact a compound and a cell,
(b) wherein the compound has the structure of Formula I: 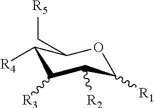
or pharmaceutically acceptable salts thereof, physiologically hydrolyzable and acceptable esters thereof, or both,
wherein R₁ is H, OH, SH, NH₂, or CH₃,
wherein R₂ is NH—R₆, wherein R₆ is H, CH₃, C₂H₅, n-propyl, C(O)CH₃, C(O)C₂H₅, C(O)n-propyl, C(O)iso-propyl, C(O)OCH₃, C(O)OC₂H₅, C(O)NH₂, or NH₂,
wherein R₃ is H, OH, SH, NH₂, or CH₃,
wherein R₄ is a hydrogen bond donor,
wherein R₅ is a hydrogen bond acceptor,
wherein the compound is not glucosamine-6-phosphate,
wherein the cell comprises a gene encoding an RNA comprising a glmS riboswitch,
wherein the compound inhibits expression of the gene by binding to the glmS riboswitch.

2. The method of claim 1, wherein R₄ is OH, SH, NH₂, NH₃+, CH₂OH, CH(OH)CH₃, CH₂CH₂OH, CH₂SH, CH(SH)CH₃, CH₂CH₂SH, CH₂NH₂, CH(NH₂)CH₃, CH₂CH₂NH₃, CO₂H, CONH₂, CONHalkyl, ═NH, ═NOH, ═NSH, ═NCO₂H, ═CH₂, CH═NH, CH═NOH, CH═NSH, CH═NCO₂H, OCH₂OH, OCH₂CH₂OH, PhOH, NHalkyl, NHNH₂, NHNHalkyl, NHCOalkyl, NHCO₂alkyl, NHCONH₂, NHSO₂alkyl, or NHOalkyl.

3. The method of claim 1, wherein R₄ is not OH when R₁ is H or OH and R₂ is NH₂ or NHCH₃.

4. The method of claim 1, wherein R₅ is OP(O)(OH)₂, OP(S)(OH)₂, OP(O)OHSH, OS(O)₂OH, or OS(O)₂SH.

5. The method of claim 1, wherein R₅ is OS(O)₂OH or OS(O)₂SH.

6. The method of claim 1, wherein R₅ is negatively charged.

7. The method of claim 1, wherein R₅ is ═O, CO₂R₉, OCO₂R₉, OCH₂OR₉, OC₂H₅OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, PO(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, and
wherein R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), —C(CH₃)₃, or —CF₃.

8. The method of claim 1, wherein R₅ is ═O, OH, OR₉, COR_{S}, CN, NO₂, tetrazole, SOR₉, N(R₉)₂, CO₂R₉, OCO₂R₉, OCH₂OR₉, OC₂H₅OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, PO₂(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, and
wherein R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), —C(CH₃)₃, or —CF₃.

9. The method of claim 1, wherein R₄ is NH₂, NH₃⁺, OH, SH, NOH, NHNH₂, NHNH₃⁺, CO₂H, SO₂OH, B(OH)₂, or imidazolium.

10. The method of claim 1, wherein R₄ is NH₂, NH₃⁺, SH, NOH, NHNH₂, NHNH₃⁺, CO₂H, SO₂OH, B(OH)₂, or imidazolium.

11. The method of claim 1, wherein the cell has been identified as being in need of inhibited gene expression.

12. The method of claim 1, wherein the cell is a bacterial cell.

13. The method of claim 1, wherein the compound kills or inhibits the growth of the bacterial cell.

14. The method of claim 1, wherein the compound and the cell are brought into contact by administering the compound to a subject.

15. The method of claim 14, wherein the compound is not a substrate for enzymes of the subject that have glucosamine-6-phosphate as a substrate.

16. The method of claim 14, wherein the compound is not a substrate for enzymes of the subject that alter glucosamine-6-phosphate.

17. The method of claim 14, wherein the compound is not a substrate for enzymes of the subject that metabolize glucosamine-6-phosphate.

18. The method of claim 14, wherein the compound is not a substrate for enzymes of the subject that catabolize glucosamine-6-phosphate.

19. The method of claim 14, wherein the cell is a bacterial cell in the subject, wherein the compound kills or inhibits the growth of the bacterial cell.

20. The method of claim 14, wherein the subject has a bacterial infection.

21. The method of claim 1, wherein the cell contains a glmS riboswitch.

22. The method of claim 12, wherein the bacteria is Bacillus or Staphylococcus.

23. The method of claim 14, wherein the compound is administered in combination with another antimicrobial compound.

24. The method of claim 1, wherein the compound inhibits bacterial growth in a biofilm.

25. A compound having the structure of Formula I: 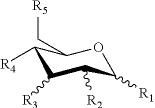
or pharmaceutically acceptable salts thereof, physiologically hydrolyzable and acceptable esters thereof, or both,
wherein R₁ is H, OH, SH, NH₂, or CH₃,
wherein R₂ is NH—R₆, wherein R₆ is H, CH₃, C₂H₅, n-propyl, C(O)CH₃, C(O)C₂H₅, C(O)n-propyl, C(O)iso-propyl, C(O)OCH₃, C(O)OC₂H₅, C(O)NH₂, or NH₂,
wherein R₃ is H, OH, SH, NH₂, or CH₃,
wherein R₄ is a hydrogen bond donor,
wherein R₅ is a hydrogen bond acceptor,
wherein the compound is not glucosamine-6-phosphate.

26. The compound of claim 25, wherein R₄ is OH, SH, NH₂, NH₃+, CH₂OH, CH(OH)CH₃, CH₂CH₂OH, CH₂SH, CH(SH)CH₃, CH₂CH₂SH, CH₂NH₂, CH(NH₂)CH₃, CH₂CH₂NH₃, CO₂H, CONH₂, CONHalkyl, ═NH, ═NOH, ═NSH, ═NCO₂H, ═CH₂, CH═NH, CH═NOH, CH═NSH, CH═NCO₂H, OCH₂OH, OCH₂CH₂OH, PhOH, NHalkyl, NHNH₂, NHNHalkyl, NHCOalkyl, NHCO₂alkyl, NHCONH₂, NHSO₂alkyl, or NHOalkyl.

27. The compound of claim 25, wherein R₄ is not OH when R₁ is H or OH and R₂ is NH₂ or NHCH₃.

28. The compound of claim 25, wherein R₅ is OP(O)(OH)₂, OP(S)(OH)₂, OP(O)OHSH, OS(O)₂OH, or OS(O)₂SH.

29. The compound of claim 25, wherein R₅ is OS(O)₂OH or OS(O)₂SH.

30. The compound of claim 25, wherein R₅ is negatively charged.

31. The compound of claim 25, wherein R₅ is ═O, CO₂R₉, OCO₂R₉, OCH₂OR₉, OC₂H₅OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, P0₂(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, and
wherein R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), —C(CH₃)₃, or —CF₃.

32. The compound of claim 25, wherein R₅ is ═O, OH, OR₉, COR_{S}, CN, NO₂, tetrazole, SOR₉, N(R₉)₂, CO₂R₉, OCO₂R₉, OCH₂OR₉, OC₂H₅OR₉, OCH₂CH₂OH, OCONHR₉, OCON(R₉)₂, CONHR₉, CON(R₉)₂, CONHCH₃OCH₃, CONHSO₂OH, CONHSO₂R₉, SO₂R₉, SO₃H, SO₂NHR₉, SO₂N(R₉)₂, PO(R₉)₂, PO₂(R₉)₂, PO(OR₉)₂, PO₂(OH)R₉, PO₂R₉N(R₉)₂, NHCH(NR₉)₂, NHCOR₉, NHCO₂R₉, NHCONHR₉, NHCON(R₉)₂, NHCONHR₉, N(COR₉)₂, N(CO₂R₉)₂, NHSO₂R₉, NR₉SO₂R₉, NHSO₂NHR₉, NR₉SO₂NH₂, NHPO(R₉)₂, NR₉PO(R₉)₂, NHPO₂OR₉, or B(OH₂)₂, and
wherein R₉ is —H, —CH₃, —C₂H₅, —CH₂CH₂CH₃, —CH(CH₃)₂, —(CH₂)₃CH₃, —CH₂CH(CH₃)₂, —CH(CH₃)CH₂(CH₃), —C(CH₃)₃, or —CF₃.

33. The compound of claim 25, wherein R₄ is NH₂, NH₃⁺, OH, SH, NOH, NHNH₂, NHNH₃⁺, CO₂H, SO₂OH, B(OH)₂, or imidazolium.

34. The compound of claim 25, wherein R₄ is NH₂, NH₃⁺, SH, NOH, NHNH₂, NHNH₃⁺, CO₂H, SO₂OH, B(OH)₂, or imidazolium.

35. The compound of claim 25, wherein the compound binds to a glmS riboswitch.

36. The compound of claim 25, wherein the compound activates a glmS riboswitch.

37. The compound of claim 25, wherein the compound is bound to a glmS riboswitch.

38. A composition comprising the compound of claim 25 and a regulatable gene expression construct comprising a nucleic acid molecule encoding an RNA comprising a glmS riboswitch operably linked to a coding region, wherein the glmS riboswitch regulates expression of the RNA, wherein the glmS riboswitch and coding region are heterologous.

39. The composition of claim 38, wherein the glmS riboswitch produces a signal when activated by the compound.

40. The composition of claim 38, wherein the riboswitch changes conformation when activated by the compound, wherein the change in conformation produces a signal via a conformation dependent label.

41. The composition of claim 38, wherein the riboswitch changes conformation when activated by the compound, wherein the change in conformation causes a change in expression of the coding region linked to the riboswitch, wherein the change in expression produces a signal.

42. The composition of claim 38, wherein the signal is produced by a reporter protein expressed from the coding region linked to the riboswitch.

43. A method comprising:
(a) testing the compound of claim 25 for inhibition of gene expression of a gene encoding an RNA comprising a glmS riboswitch, wherein the inhibition is via the glmS riboswitch,
(b) inhibiting gene expression by bringing into contact a cell and a compound that inhibited gene expression in step (a),
wherein the cell comprises a gene encoding an RNA comprising the glmS riboswitch, wherein the compound inhibits expression of the gene by binding to the glmS riboswitch.

44. The atomic structure of a natural glmS-responsive riboswitch comprising an atomic structure comprising the atomic coordinates listed in Table 2.

45. The atomic structure of a natural glmS-responsive riboswitch comprising an atomic structure comprising the binding pocket atomic structure.

46. A method of identifying a compound that interacts with a riboswitch comprising:
(a) modeling the atomic structure of claim 44 with a test compound; and
(b) determining if the test compound interacts with the riboswitch.

47. The method of claim 46, wherein determining if the test compound interacts with the riboswitch comprises determining a predicted minimum interaction energy, a predicted binding constant, a predicted dissociation constant, or a combination, for the test compound in the model of the riboswitch.

48. The method of claim 46, wherein determining if the test compound interacts with the riboswitch comprises determining one or more predicted bonds, one or more predicted interactions, or a combination, of the test compound with the model of the riboswitch.

49. The method of claim 46, wherein atomic contacts are determined in step (b), thereby determining the interaction of the test compound with the riboswitch.

50. The method of claim 49, further comprising the steps of:
(c) identifying analogs of the test compound;
(d) determining if the analogs of the test compound interact with the riboswitch.

51. A method of killing or inhibiting the growth of bacteria, comprising contacting the bacteria with an analog identified by the method of claim 50.

52. A method of killing or inhibiting the growth of bacteria, comprising contacting the bacteria with a compound identified by the method of claim 46.

53. The method of claim 46, wherein a gel-based assay is used to determine if the test compound interacts with the riboswitch.

54. The method of claim 46, wherein a chip-based assay is used to determine if the test compound interacts with the riboswitch.

55. The method of claim 46, wherein the test compound interacts via van der Waals interactions, hydrogen bonds, electrostatic interactions, hydrophobic interactions, or a combination.

56. The method of claim 46, wherein the riboswitch comprises an RNA cleaving ribozyme.

57. The method of claim 46, wherein a fluorescent signal is generated when a nucleic acid comprising a quenching moiety is cleaved.

58. The method of claim 46, wherein molecular beacon technology is employed to generate the fluorescent signal.

59. The method of claim 46, wherein the method is carried out using a high throughput screen.
